(19) United States
(12) Patent Application Publication
(10) Pub. No.: US 2015/0128300 A1
(43) Pub. Date: May 7, 2015

(54) METHODS AND COMPOSITIONS FOR GENERATING CONDITIONAL KNOCK-OUT ALLELES

(71) Applicant: GENENTECH, INC., South San Francisco, CA (US)

(72) Inventors: Soren Warming, Millbrae, CA (US); Keith R. Anderson, Kensington, CA (US)

(21) Appl. No.: 14/407,869

(22) PCT Filed: Jun. 12, 2013
(86) PCT No.: PCT/US13/45382

§ 371 Date: Dec. 12, 2014

Related U.S. Application Data
(60) Provisional application No. 61/658,670, filed on Jun. 12, 2012.

(51) Int. Cl. C12N 15/90

(52) U.S. Cl. CPC C12N 15/907
USPC 800/14; 435/462; 800/21; 435/196; 536/23.5; 435/325

(57) ABSTRACT

The disclosure provides methods and compositions for generating conditional knock-out alleles using donor constructs together with sequence-specific nucleases to generate conditional knock-out alleles. Specifically, the donor construct comprises a 5′ homology region, a 5′ recombinase recognition site, a donor sequence, a 3′ recombinase recognition site, and a 3′ homology region. Further disclosed are the donor sequences each comprises a target sequence having at least one neutral mutation. Different sequence-specific nucleases can be used with the donor constructs are further disclosed.

### CROSS REFERENCE TO RELATED APPLICATION

This application claims the benefit of U.S. Provisional Application No. 61/658,670, filed Jun. 12, 2012, the disclosure of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Jun. 12, 2013, is named P4905R1WO_PCTSequenceListing.txt and is 49,214 bytes in size.

### FIELD OF THE INVENTION

The present invention concerns novel methods of producing genetically engineered conditional knock out alleles.

### BACKGROUND

Selective inhibition or enhancement of individual gene expression has greatly assisted the study of gene function in vitro and in vivo. Gene targeting of murine embryonic stem (ES) cells using homologous recombination is a well-established method for manipulating the murine genome and has allowed creation of null or “knock-out” mice with respect to a gene under investigation. More recently, conditional or inducible knock-out technology has advanced the study of genes that, when deleted systemically, result in embryonic or perinatal lethality (e.g., Lakso, M. et al., Proc. Natl. Acad. Sci. USA 89:6232-36 (1992); Jacks, T. et al., Nature 359:295-300 (1992)). Conditional knock-out mice can also be used to study the effects of selectively deleting a gene in a particular tissue, while leaving its function intact in other tissues. However, conventional methods for creating conditional knock-out animals are laborious, inefficient and require the availability of embryonic stem cells.

Engineered sequence-specific nucleases have been used to create knock-out alleles. Examples of such sequence-specific endonucleases include zinc finger nucleases (ZFNs), which are composed of sequence-specific DNA binding domains fused to an endonuclease effector domain (Porteus, M. H. and Caroll, D., Nat. Biotechnol. 23, 967-973 (2005)). Another example of sequence-specific nucleases are transcription activator-like effector nucleases (TALENs), which are composed of a nuclease domain fused to TAL effector proteins (Miller, J. C. et al., Nat. Biotechnol. 29, 143-148 (2011); Cermak, T. et al., Nucleic Acid Res. 39, e82 (2011)). Sequence-specific endonucleases are modular in nature, and DNA binding specificity is obtained by arranging one or more modules. For example, zinc finger domains in ZFNs each recognize three base pairs (Bibikova, M. et al., Mol. Cell. Biol. 21, 289-297 (2001)), whereas individual TAL domains in TALENs each recognize one base-pair via a unique code (Boch, J. et al., Science 326, 1509-1512 (2009).) Another example of sequence-specific nucleases includes RNA-guided DNA nucleases, e.g., the CRISPR/Cas system.

ZFNs, TALENs and most recently CRISPR/Cas mediated gene editing have been used to efficiently and directly generate gene knock-out alleles (Geurts, A. M. et al., Science 325, 433 (2009); Mashimo, T. et al., PLoS ONE 5, e8870 (2010); Carbery, I. D. et al., Genetics 186, 451-459 (2010); Tesson, L., et al., Nat. Biotech. 29, 695-696 (2011)). The knock-out alleles are thought to be produced by an error-prone non-homologous end joining (NHEJ) of the endonuclease-mediated double-strand break (DSB).

Recently, ZFNs were successfully used for targeted insertion (knock-in) of a reporter gene by homologous recombination of the targeted chromosomal locus with a donor DNA in both mouse and rat (Meyer, M., et al., Proc. Natl. Acad. Sci. USA 107, 15022-15026 (2010); Cui, X. et al., Nat. Biotechnol. 29(1), 64-67 (2010)). The sequence-specific insertion of the donor sequence has been proposed to occur via a synthesis-dependent strand annealing (SDSA) model of double-strand break repair by homologous recombination between the donor and the locus at which the double-strand break occurred (Moehle, E. A. et al., Proc Natl Acad Sci USA 104, 3055-3060 (2007)). According to this model, after endonuclease-mediated double-strand break and strand resection, the single-stranded chromosome ends anneal to the homology regions present on the donor DNA followed by synthesis using the donor insert as template.

Despite these advances, a need in the art remains for new methods to create conditional knock-out alleles and to expand this technology to other species. The present invention fulfills this need and provides other benefits.

### SUMMARY

The present invention relates to novel methods and compositions for generating conditional knock-out alleles. Specifically, the present invention relates to using specific donor constructs together with sequence-specific nucleases to generate conditional knock-out alleles.

In one aspect, a method of generating a conditional knock-out allele in a cell comprising a target gene is provided. The method comprises the steps of:
1. introducing into the cell a donor construct, wherein the donor construct comprises a 5′ homology region, a 5′ recombinase recognition site, a donor sequence, a 3′ recombinase recognition site, and a 3′ homology region, wherein the donor sequence comprises a target sequence having at least one neutral mutation; and
2. introducing into the cell a sequence-specific nuclease that cleaves a sequence within the target gene, thereby producing a conditional knock-out allele in the cell.

In certain embodiments, the sequence-specific nuclease is a zinc finger nuclease (ZFN), a ZFN dimer, a transcription activator-like effector nuclease (TALEN), or a RNA-guided DNA endonuclease. In certain embodiments, the sequence-specific nuclease cleaves the target gene only once. In certain embodiments, the sequence-specific nuclease is introduced into the cell as a protein, mRNA, or cDNA.

In certain embodiments, the recombinase recognition site is a loxP site, a rox site or an frt site. In certain embodiments, the donor sequence comprises one, two, three, four, five, six, seven, eight, nine, ten, eleven, or twelve neutral mutations. In certain embodiments, the homology between the donor sequence and the target sequence is 51-99%. In certain embodiments the homology between the donor sequence and the target sequence is 78%. In certain embodiments, the donor construct comprises the sequence shown in FIG. 4A or FIG. 4B. In certain embodiments, the 5′ homology region comprises at least 1.1 kb and wherein the 3′ homology region comprises at least 1 kb. In certain embodiments, the target gene is Lrp5.

In a further embodiment, the cell is a mammalian cell. In certain embodiments, the mammalian cell a mouse, rat, rabbit, hamster, cat, dog, sheep, horse, cow, monkey or human cell. In certain embodiments, the cell is from a non-human animal. In certain embodiments, the cell is a somatic cell, a zygote or a pluripotent stem cell.

In a further aspect, a method of generating a conditional knock-out animal is provided, the method comprising the steps of:
1. introducing a donor construct into a cell comprising a target gene, wherein the donor construct comprises a 5′ homology region, a 5′ recombinase recognition site, a donor sequence, a 3′ recombinase recognition site, and a 3′ homology region, wherein the donor sequence comprises a target sequence having at least one neutral mutation;
2. introducing a sequence-specific nuclease into the cell, wherein the nuclease cleaves the target gene; and
3. introducing the cell into a carrier animal to produce the conditional knock-out animal from the cell.

In some embodiments, the animal is a mouse, rat, rabbit, hamster, guinea pig, dog, sheep, pig, horse, cow or monkey. In certain embodiments, the cell is from a non-human animal. In some embodiments, the cell is a zygote or a pluripotent stem cell.

In a further aspect, a method of generating a knock-out animal is provided, the method comprising the steps of:
1. introducing a donor construct into a zygote comprising a target gene, wherein the donor construct comprises a 5′ homology region, a 5′ recombinase recognition site, a donor sequence, a 3′ recombinase recognition site, and a 3′ homology region, wherein the donor sequence comprises a target sequence having at least one neutral mutation;
2. introducing a sequence-specific nuclease into the zygote, wherein the nuclease cleaves the target gene;
3. introducing the zygote into a carrier animal to produce a conditional knock-out animal from the zygote; and
4. breeding the conditional knock-out animal with a transgenic animal having a transgene encoding a recombinase that catalyzes recombination at the 5′ and 3′ recombinase recognition sites, thereby producing the knock-out animal.

In certain embodiments, the recombinase recognition site is a loxP site and the recombinase is Cre recombinase. In certain embodiments, the recombinase recognition site is an frt site and the recombinase is flippase. In certain embodiments, the recombinase recognition site is a rox site and the recombinase is Dre recombinase. In certain embodiments, the transgene encoding the recombinase is under the control of a tissue-specific promoter.

In a further aspect of the invention, a composition for generating a conditional knock-out allele of a target gene is provided, comprising:
1. a donor construct comprising a 5′ homology region, a 5′ recombinase recognition site, a donor sequence, a 3′ recombinase recognition site, and a 3′ homology region, wherein the donor sequence comprises a target sequence having at least one neutral mutation; and
2. a sequence-specific nuclease that recognizes the target gene.

In certain embodiments, the sequence-specific nuclease is a ZFN, a ZFN dimer, a ZFNickase, a TALEN, or a RNA-guided DNA endonuclease. In certain embodiments, the recombinase recognition site is a loxP site, an frt site or a rox site.

In a further aspect of the invention, a donor construct comprising the sequence shown in FIG. 4A (SEQ ID NO: 30), FIG. 4B (SEQ ID NO: 31), or FIG. 14C (SEQ ID NOS: 44-46) is provided.

In a further aspect of the invention, a cell comprising the donor construct comprising the sequence shown in FIG. 4A (SEQ ID NO: 30), FIG. 4B, or FIG. 14C (SEQ ID NOS: 44-46) is provided. In certain embodiment, the cell is a mammalian cell. In certain embodiments, the mammalian cell a mouse, rat, rabbit, hamster, cat, dog, sheep, horse, cow, monkey or human cell. In certain embodiments, the cell is from a non-human animal. In certain embodiments, the cell is a somatic cell, a zygote or a pluripotent stem cell.

In a further aspect of the invention, a non-human conditional knock-out animal prepared according to the method described herein is provided.

### BRIEF DESCRIPTION OF THE FIGURES

FIG. 1 shows the distribution of ZFN-mediated mutant Lrp5 alleles in live-born mice. The size of deletions and insertions are indicated in base pairs on the x-axis. Compound KO: animals with two independent mutant alleles of the same gene and no detectable wildtype allele of the gene; Multiple allele: chimeric animals carrying more than two alleles; SKG->WTD: deletion of

TCCAAGGGT

(ZFN cut site is underlined).

FIGS. 2A-E show vascular phenotypes of 2-month-old mice with compound in frame and out-of-frame deletions in Lrp5. 542: chimeric functional heterozygous mouse (control) that carried an allele with a 3 bp in-frame deletion that appeared to be silent and an allele with a 1 bp out-of-frame deletion; 495: mouse that carried a 4 bp out-of-frame deletion allele and a 1 bp out-of-frame deletion allele; 519: mouse that carried a 29 bp out-of-frame deletion allele and a 17 bp out-of-frame deletion allele; 555: functional heterozygous mouse that carried a 3 bp in-frame deletion allele and a 1 bp out-of-frame deletion allele and is a functional heterozygote; FA: fluorescent angiography; IB4: isolectin B4; NFL: nerve fiber layer; IPL: inner plexiform layer; OPL: outer plexiform layer.

FIGS. 3A-B show conditional knock-out alleles obtained from co-microinjection or co-electroporation of Lrp5 exon2 ZFN and donor plasmid. FIG. 3A depicts a schematic of double-strand break repair by synthesis-dependent strand annealing. Arrow heads represent recombinase recognition sites; large arrow in Step 1 represents the target sequence; large arrow with asterisks represents the donor sequence; asterisks represent neutral mutations; half arrows indicate primer positions. FIG. 3B depicts the results of a polymerase chain reaction (PCR) analysis of DNA isolated from tail samples of pups (left panel) or ES cells (right panel). The respective primer pairs used for the analysis are indicated to the left (primer positions are as depicted in FIG. 3A).

FIGS. 4A-C show the donor sequences (SEQ ID NOS: 30-32, respectively, in order of appearance) that were used in plasmids in the correct orientation and with the sequences flanking the inserts.

FIGS. 5A-B show a sequence alignment of the three Lrp5 CKO DNA donors from 5′ loxP to 3′ loxP sites (SEQ ID NOS 33-35, respectively, in order of appearance). Uppercase bold letters indicate loxP sites; lowercase letters indicate intron sequences; uppercase letters indicate exon 2 (wild type or modified) sequences; dashed line boxes indicate ZFN binding sites; solid line boxes indicate silent mutations; underlined letters indicate the sequence at which the wild type exon 2 is cleaved by the ZFN.

FIGS. 6A-E show normal retinal phenotypes of mice carrying a codon-modified Lrp5 conditional knock-out allele. FIGS. 6A-D depict confocal projections of retinal whole mounts stained with isolectin B4 (scale bars: 50 μm). FIG. 6E depicts retinal cross sections of the opposite eyes to those depicted in FIGS. 6A-D, stained with IB4, MECA32, and DAPI. Arrows point to example staining as indicated. +/+: wild type control; KO/KO: Lrp5 homozygous knock out; KO/+: Lrp5 heterozygous knock out; CKO/KO: Lrp5 conditional knock out/Lrp5 knock-out compound heterozygous; IB4: isolectin B4; NFL: nerve fiber layer; IPL: inner plexiform layer; OPL: outer plexiform layer.

FIGS. 7A-D show a graphic representation of possible mechanism that produced each of the observed donor-derived Lrp5 alleles. Primers that bind to the resulting alleles are indicated. Neutral mutations are indicated by asterisks.

FIG. 8 depict the results of a SURVEYOR Assay following introduction of either zinc finger pairs (pZFN1+pZFN2) or Cas9 (+pRK5-hCas9) together with a guide RNA targeting Lrp5 exon 2 (p_gRNA T2, p_gRNA T5 or p_gRNA T7) or a control plasmid (PMAXGFP) into NIH/3T3 cells or Hepa1-6 cells.

FIGS. 9A-B illustrate a summary of gRNA/Cas9 mutation rates (FIG. 9A) and deletion sizes (FIG. 9B) at the Lrp5 exon 2 genomic locus in Hepa1-6 murine hepatoma cells. The cells received a gRNA targeting Lrp5 together with either mRNA (Cas9 mRNA+gRNA T2, solid bars) or a plasmid (Cas9 plasmid+gRNA T2, clear bars), or two plasmids encoding zink finger pairs targeting exon2 of Lrp5 (ZFN plasmid, grey bars).

FIG. 10 depicts the result of PCR analysis using a forward primer specific for the COexon2 sequence and a reverse primer outside of the homology arm in the genomic locus to identify integration of the donor exon in the Lrp5 locus. Murine Hepa1-6 cells received plasmid (pRK5-hCas9) or mRNA (hCas9 mRNA) encoding Cas9 together with either the guide RNA alone (p_gRNA T2), the guide RNA and the donor plasmid (p_gRNA T2+p_donor1) or a control plasmid (PMAXGFP). Some cells received the donor together with the Lrp5 zink finger pair (pZFN1+pZFN2+p_donor1).

FIG. 11 depicts the result of PCR analysis using primers that detect 5′ (top, primers P9 and P10) and 3′ (bottom, primers P11 and P12) loxP site integration in the Lrp5 genomic locus. The treatment groups are as described in FIG. 10. DNA from a heterozygous Lrp5 conditional knock out (mouse CKO/wt) was used as positive control.

FIG. 12 depicts the results of a SURVEYOR Assay following introduction of Cas9 (p_hCas9) together with a guide RNA and respective donor construct targeting Lrp5 (Lrp5 exon 2; p_gRNA T7+p_Lrp5_donor1), Usp10 (Usp10 exon3; p_gRNA T1+p_Usp10_donor1) or Notch3 (Notch3 exon3; p_gRNA T1+p_Notch3_donor1) into Hepa1-6 cells.

FIG. 13 depicts the result of PCR analysis using primers that detect 5′ loxP site integration in the Nnmt exon2 genomic locus (left panel, primers P26 and P27) or 3′ loxP site integration in the Notch3 exon3 genomic locus (right panel, primers P25 and P28) following Cas9/gRNA and donor administration.

FIG. 14A-D show the sequences (SEQ ID NOS: 36-46, respectively, in order of appearance) for Cas9/CRISPR targeting of mouse Lrp5, Usp10, Nnmt, and Notch3 genomic loci. Sequences for guide RNA (gRNA) sequences specific for Lrp5, Usp10, Nnmt, and Notch3 and donor plasmid sequences for Usp10, Nnmt, and Notch3 are depicted. In addition, Cas9 cDNA sequence for mammalian expression and in vitro transcription (mRNA) are shown.

### DETAILED DESCRIPTION OF EMBODIMENTS OF THE INVENTION

### I. Definitions

For purposes of interpreting this specification, the following definitions will apply and whenever appropriate, a term used in the singular will also include the plural and vice versa. In the event that any definition set forth below conflicts with any document incorporated herein by reference, the definition set forth below shall control.

The term “donor construct,” as used herein, refers, unless specifically indicated otherwise, to a polynucleotide that comprises a 5′ homology region, a 5′ recombinase recognition site, a donor sequence, a 3′ recombinase recognition site, and a 3′ homology region. The donor construct can further include additional sequences, such as sequences that support propagation of the donor construct or selection of cells harboring the construct.

The term “donor sequence,” as used herein, refers, unless specifically indicated otherwise, to a nucleic acid having a sequence that comprises a target sequence having at least one neutral mutation compared to a portion of the sequence of the target gene. As such, the donor sequence comprises a nucleic acid that encodes a polypeptide that is functionally substantially similar to or indistinguishable from that encoded by the portion of the target gene. Consequently, the donor sequence can replace the cognate portion of the target gene at its position in the target gene without substantially changing the functional properties of the protein encoded by the target gene. The donor sequence can comprise certain non-coding sequences, such as intronic or regulatory sequences.

The term “homology region,” as used herein, refers, unless specifically indicated otherwise, to a nucleic acid in the donor construct that is homologous to a nucleic acid flanking a target sequence.

The term “recombinase recognition site,” as used herein, refers, unless specifically indicated otherwise, to a nucleic acid in a donor construct having a sequence that is recognized by a recombinase.

The term “recombinase,” as used herein, refers, unless specifically indicated otherwise, to an enzyme that recognizes specific polynucleotide sequences (recombinase recognition sites) that flank an intervening polynucleotide and catalyzes a reciprocal strand exchange, resulting in inversion or excision of the intervening polynucleotide.

The term “target gene,” as used herein, refers, unless specifically indicated otherwise, to a nucleic acid encoding a polypeptide within a cell.

The term “target sequence,” as used herein, refers, unless specifically indicated otherwise, to a portion of the target gene, e.g., one or more of the exon sequences of the target gene, intronic sequences, or regulatory sequences of the target gene, or a combination of exon and intron sequences, intron and regulatory sequences, exon and regulatory sequences, or exon, intron, and regulatory sequences of the target gene.

The term “sequence-specific endonuclease” or “sequence-specific nuclease,” as used herein, refers, unless specifically indicated otherwise, to a protein that recognizes and binds to a polynucleotide, e.g., a target gene, at a specific nucleotide sequence and catalyzes a single- or double-strand break in the polynucleotide.

The term “RNA-guided DNA nuclease” or “RNA-guided DNA nuclease” or “RNA-guided endonuclease,” as used herein, refers, unless specifically indicated otherwise, to a protein that recognizes and binds to a guide RNA and a polynucleotide, e.g., a target gene, at a specific nucleotide sequence and catalyzes a single- or double-strand break in the polynucleotide.

The term “conditional knock-out allele,” as used herein, refers, unless specifically indicated otherwise, to an allele comprising a polynucleotide sequence that is flanked by recombinase recognition sites but produces a phenotype that is indistinguishable from that produced by the cognate wild type allele.

The term “neutral mutation,” as used herein, refers, unless specifically indicated otherwise, to a mutation in a donor sequence that reduces overall homology between the donor sequence and the target sequence but leaves the donor sequence capable of encoding a functional polypeptide. Examples of neutral mutations include silent mutations, i.e., mutations that alter the nucleotide sequence but not the encoded polypeptide sequence. Examples of neutral mutations also include conservative mutations, such as point mutations (e.g., substitutions), insertions and deletions, i.e., mutations that alter the nucleotide sequence and the encoded polypeptide sequence but that do not substantially alter the function of the resulting polypeptide. Examples of conservative substitution mutations are shown in Table 8. Neutral mutations can also include combinations of silent mutations, combinations of conservative mutations, or combinations of silent and conservative mutations.

The term “animal,” as used herein, refers, unless specifically indicated otherwise, to any non-human animal, including, but not limited to, domesticated animals (e.g., cows, sheep, cats, dogs, and horses), primates (e.g., non-human primates such as monkeys), rabbits, fish, rodents (e.g., mice, rats, hamsters, guinea pigs), and non-vertebrates (e.g., Drosophila melanogaster and Caenorhabditis elegans).

An “isolated” nucleic acid refers, unless specifically indicated otherwise, to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a protein” refers, unless specifically indicated otherwise, to one or more nucleic acid molecules encoding a protein (or fragments thereof), including such nucleic acid molecule(s) in a single vector or separate vectors, and such nucleic acid molecule(s) present at one or more locations in a host cell.

The term “sequence homology,” as used herein with respect to the donor and target gene polynucleotide sequences, is defined as the percentage of nucleotide residues in a donor sequence that are identical to the nucleotide residues in the target gene sequence, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared.

### II. Embodiments of the Invention

The invention relates, in part, to the recognition and solution of technical challenges associated with creating conditional knock-out alleles using sequence-specific endonucleases in combination with a recombinase recognition sequence-flanked donor sequence. This process relies on targeting specific sequences of nucleic acid molecules, such as chromosomes, with endonucleases that recognize and bind to such sequences and induce a double-strand break in the nucleic acid molecule. The double strand break is repaired either by an error-prone non-homologous end joining or by homologous recombination. If a template for homologous recombination is provided in trans, the double-strand break can be repaired using the provided template. The initial double strand break increases the frequency of targeting by several orders of magnitude, compared to conventional homologous recombination-based gene targeting. In principle, this method could be used to insert any sequence at the site of repair so long as it is flanked by appropriate regions homologous to the sequences near the double-strand break. However, this approach is associated with certain challenges when applied to creating conditional knock-out alleles. Conditional knock-out alleles typically include certain recombinase recognition sequences, such as loxP sites, that flank the gene or portions of the gene but leaves its function intact, such that the conditional knock-out allele produces functional polypeptides substantially similar to the unmodified allele but that can be rendered non-functional at a certain time or within certain tissues by the presence of the recombinase recognizing the recognition sequences.

A first challenge associated with the approach described above to create conditional knock-out alleles resides in the fact that, following the double-strand break catalyzed by the sequence-specific endonuclease, undesirable recombination can occur between the donor exon and the chromosomal (target) exon, instead of the homology regions outside of the recombinase recognition sequence-flanked donor, because of their sequence identity with respect to each other. This will result in alleles that lack one or both recombinase recognition sequences. A second challenge resides in the fact that the sequence-specific endonuclease can recognize and cleave not only the target gene but also the donor exon before it can serve as a template for repair. The methods and compositions described herein provide a solution to these challenges.

A. Exemplary Methods

In various aspects of the invention, methods of generating a conditional knock-out allele in a cell comprising a target gene are provided. The method comprises the steps of introducing into the cell having a target gene a donor construct and a sequence-specific nuclease that cleaves a sequence within the target gene but does not inhibit function of the donor construct, thereby producing a conditional knock-out allele in the cell. These and further aspects of the invention are described below.

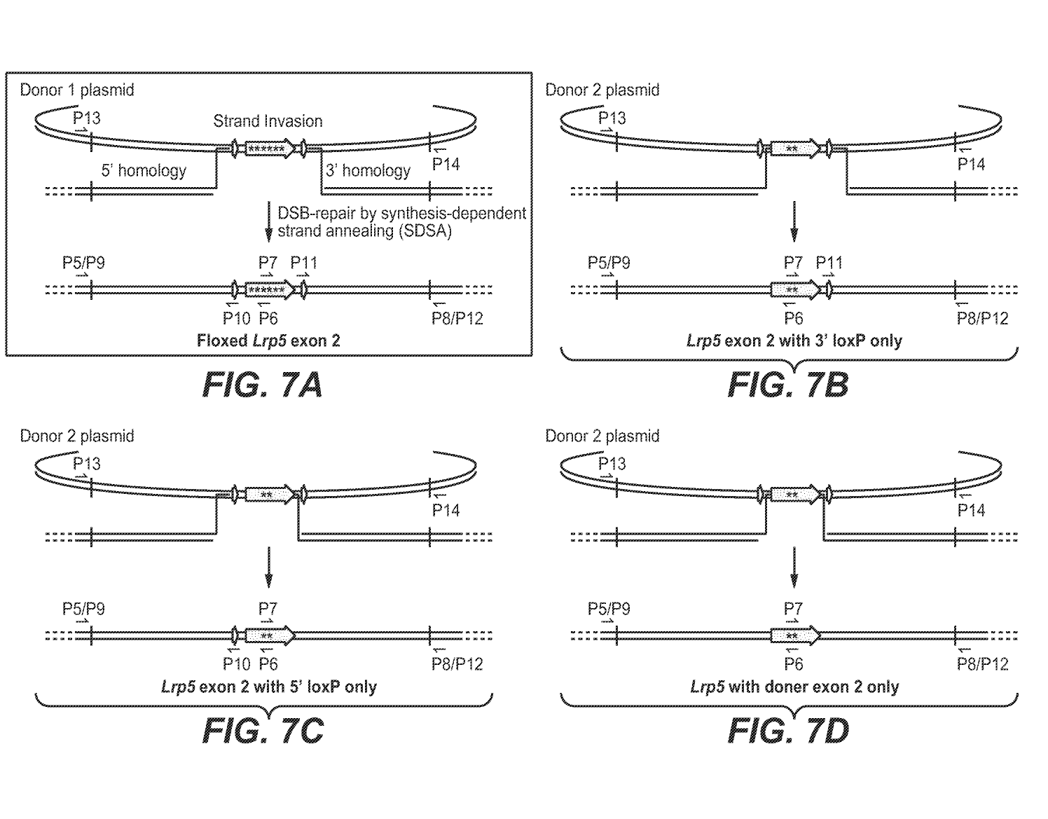

In a particular aspect of the invention, a conditional knock-out allele is produced in a cell comprising a target gene by introducing into the cell a donor construct that comprises a 5′ homology region, a 5′ recombinase recognition site, a donor sequence, a 3′ recombinase recognition site, and a 3′ homology region. The donor sequence comprises the sequence of a target sequence having at least one neutral mutation. In certain embodiments, the donor sequence and the target sequence are identical except for the at least one neutral mutation. A neutral mutation means any mutation in the nucleotide sequence of the donor sequence that reduces homology between the donor sequence and the target sequence but leaves the coding potential of the donor for a functional polypeptide intact. The neutral mutation decreases the number of undesired homologous recombination events, compared to a wild type sequence, between the donor sequence and the target sequence that do not result in a conditional knock-out allele (FIG. 7B, C, D). In some embodiments, the neutral mutation also abrogates binding of the sequence-specific nuclease to the donor sequence.

Examples of neutral mutations include silent mutations, i.e., mutations that alter the nucleotide sequence but not the encoded polypeptide sequence. Neutral mutations also include conservative mutations, i.e., mutations that alter the nucleotide sequence and the encoded polypeptide sequence but that do not substantially alter the function of the resulting polypeptide. This is the case, for example, when one amino acid is substituted with another amino acid that has similar properties (size, charge, etc.). For example, Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

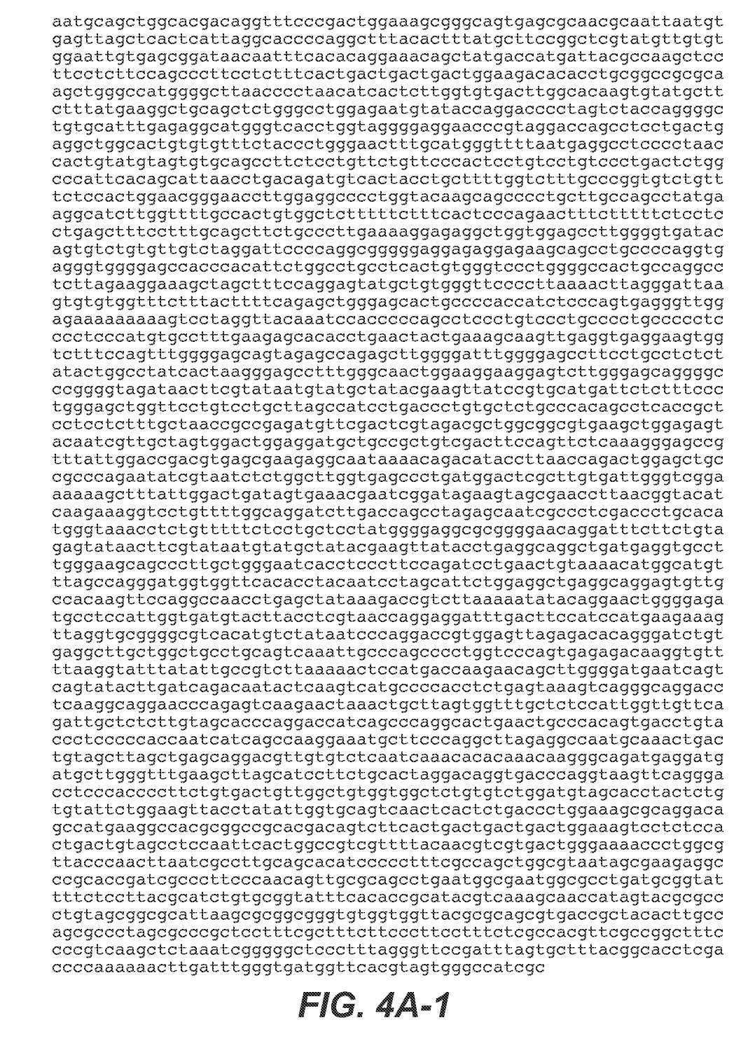

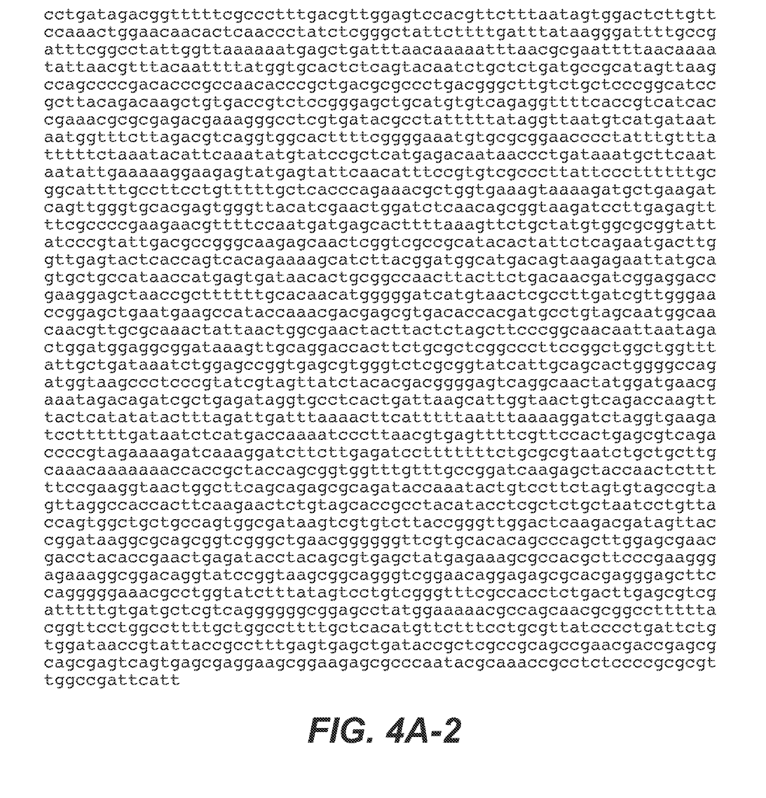

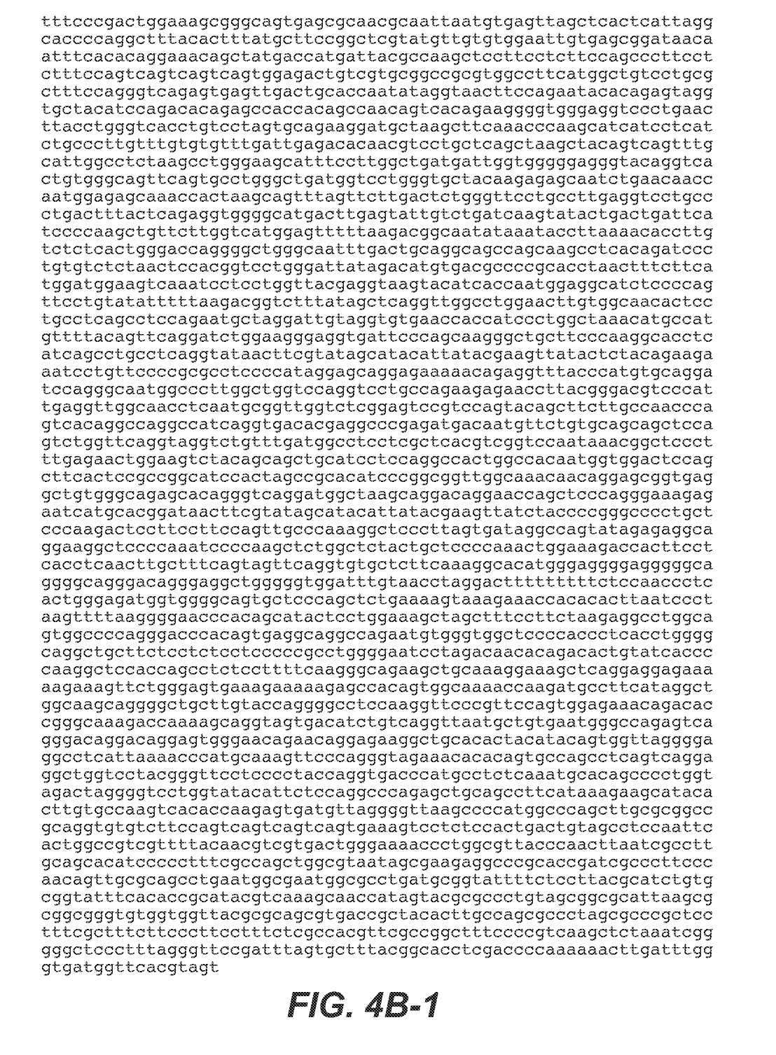

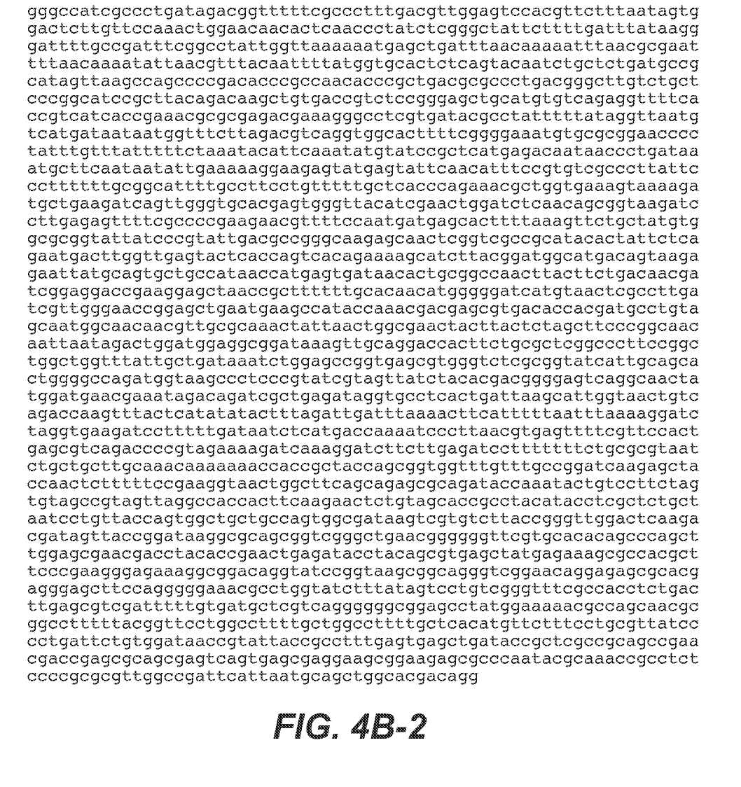

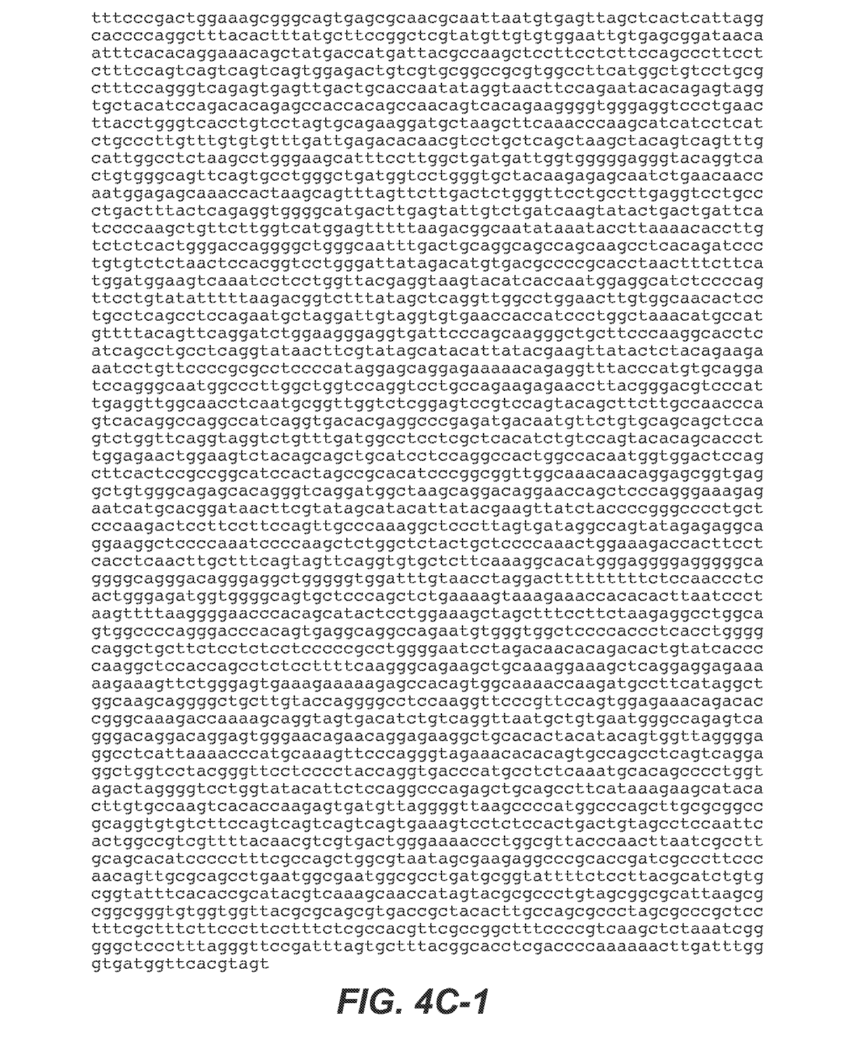

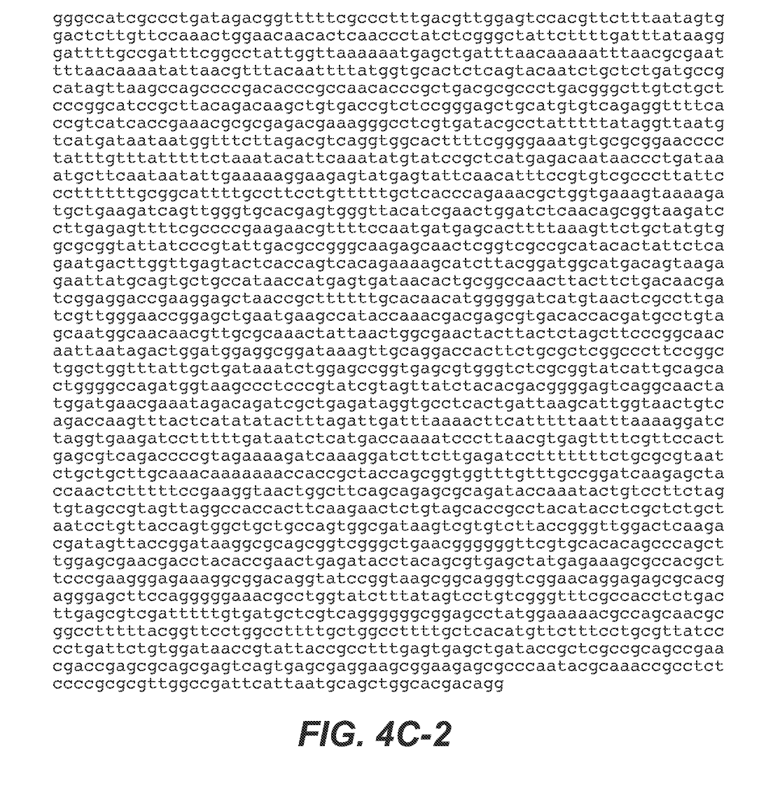

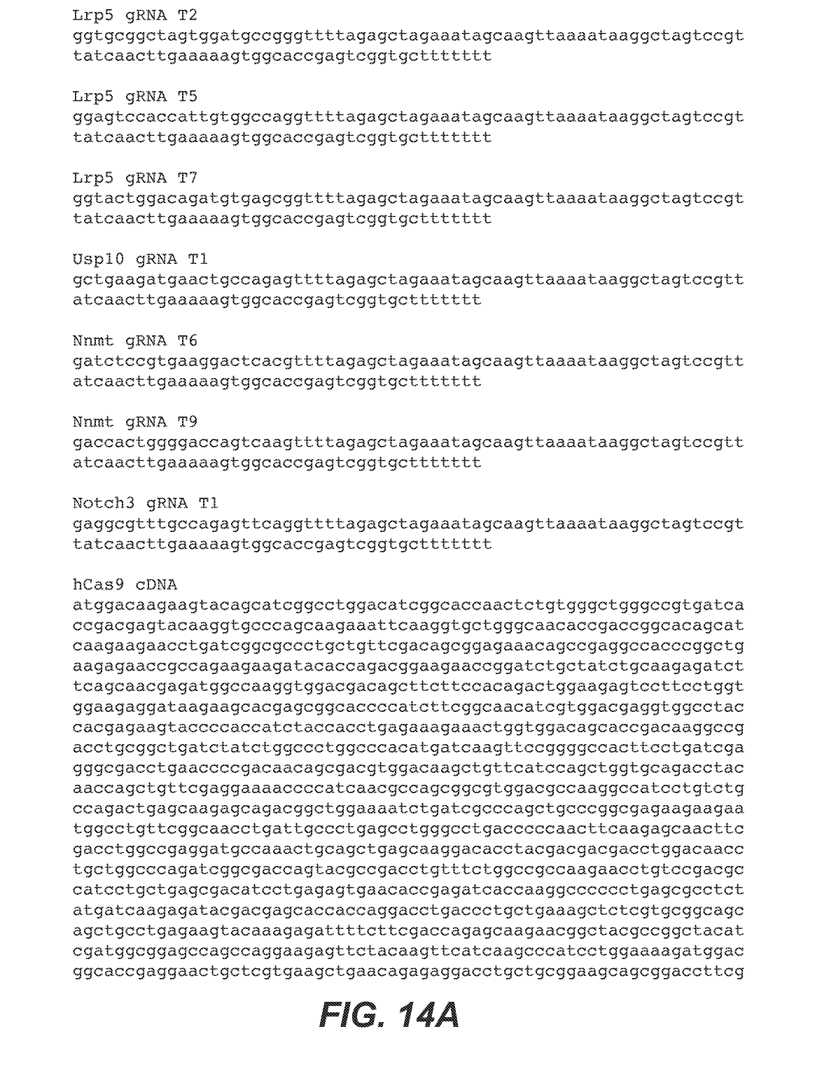

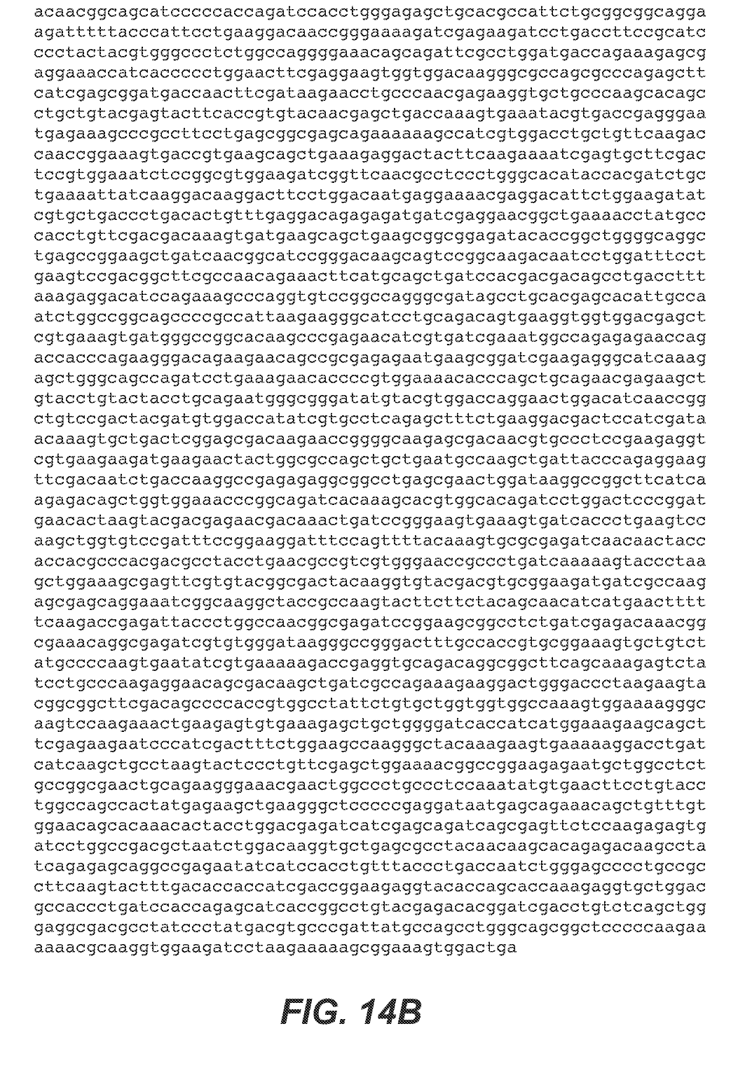

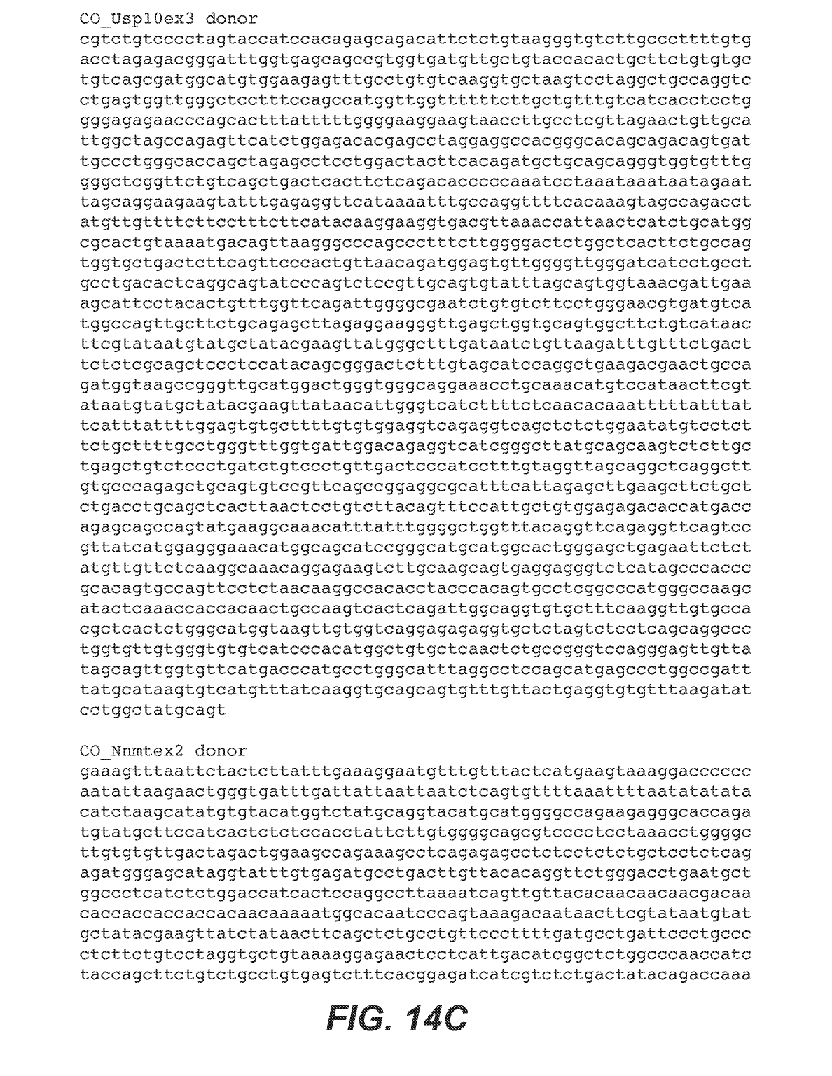

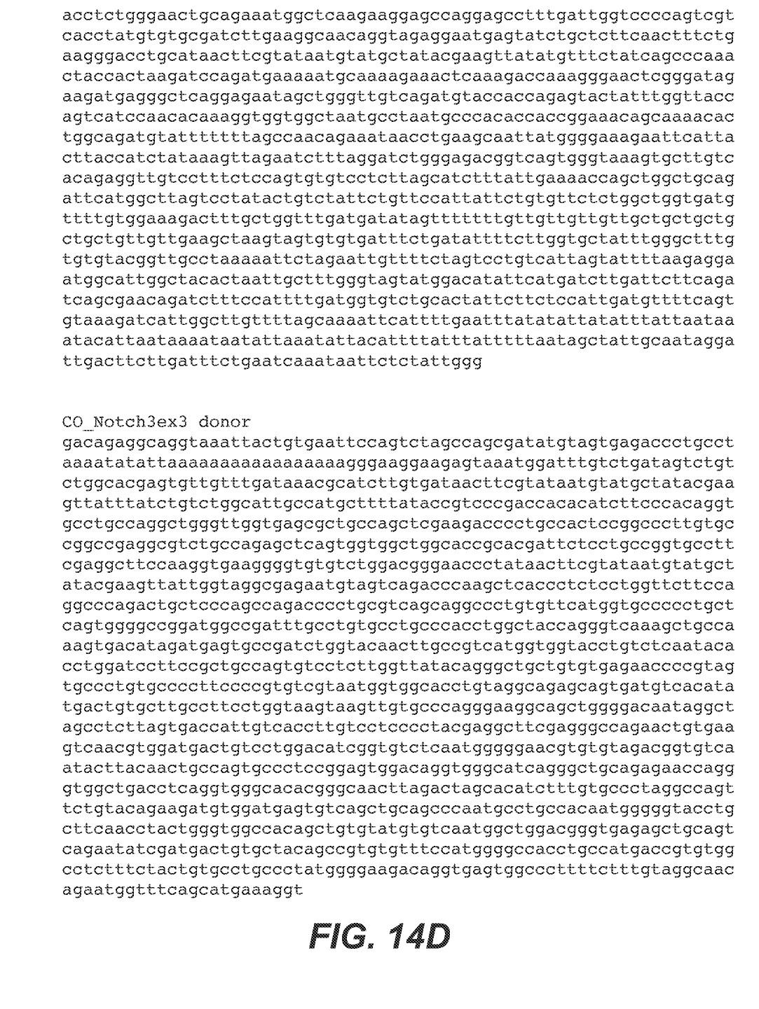

Examples of conservative mutations are shown in Table 8. In certain embodiments, the donor sequence comprises 1, 2, 3, 4, 5, 6, 7, 8, 9, 10, 15, 20, or 50 silent mutations. In certain embodiments, the homology between the donor sequence and the target sequence is 99%, 98%, 95%, 90%, 85%, 80%, 78%, 75%, 70%, 65%, 60%, 55%, or 50%. In certain embodiments, the sequence homology between donor and target sequence is less than 50%. Any number of neutral mutations can be introduced that reduce or inhibit the number of homologous recombination events between the donor sequence and the target sequence (FIG. 7B-D), rather than between the homologous regions and their cognate sequence on the targeted molecule, but maintain the ability of the donor sequence to encode a functional polypeptide. In certain embodiments, the donor comprises the sequence shown in FIG. 4A (SEQ ID NO: 30), FIG. 4B (SEQ ID NO: 31), or FIG. 14C (SEQ ID NOS: 44-46). In certain embodiments, at least one neutral mutation abrogates binding of the sequence-specific nuclease to the donor sequence. In certain embodiments, several neutral mutations are spaced along the length of the donor sequence to reduce the number of consecutive unmodified base pairs to less than 20-100 base pairs at any position in the donor sequence.

Because the mutations within the donor sequence are neutral, the donor sequence encodes a polypeptide that is functionally substantially similar to or indistinguishable from that encoded by the target sequence. The functionality of a peptide or protein can be assessed by methods well-known in the art, such as functional assays, enzymatic assays, and biochemical assays. The donor sequence can replace the target sequence at its position in the target gene without substantially altering the functional properties of the polypeptide encoded by the target gene. However, once integrated in the target gene, subsequent removal of the donor sequence from the target gene can result in altered, reduced or loss of function of the polypeptide encoded by the target gene.

Within the donor construct, the donor sequence is flanked 5′ and 3′ by recombinase recognition sites. These recombinase recognition sites are nucleic acid sequences within the donor construct that are recognized by a recombinase that subsequently catalyzes recombination at the recombination recognition sites. Sequence-specific recombination is well-known in the art and includes recombinase-mediated sequence-specific cleavage and ligation of a polynucleotide flanked by the recombinase recognition sites. Examples of recombinase recognition sites include loxP (locus of X-over P1) sites (Hoess et al., Proc. Natl. Acad. Sci. USA 79:3398-3401 (1982)), frt sites (McLeod, M., Craft, S. & Broach, J. R., Molecular and Cellular Biology 6, 3357-3367 (1986)) and rox sites (Sauer, B. and McDermott, J., Nucleic Acids Res 32, 6086-6095 (2004).).

The 5′ homology region is located 5′ or “upstream” of the 5′ recombinase recognition site and is homologous to a nucleic acid flanking the target sequence in its nucleotide context. Similarly, the 3′ homology region is located 3′ or “downstream” of the 3′ recombinase recognition site and is homologous to a nucleic acid flanking the target sequence. In one embodiment, the homology regions are more than 30 bp, preferably several kb in length. For example, the homology regions can be 50 bp, 100 bp, 200 bp, 300 bp, 500 bp, 800 bp, 1 kb, 1.1 kb, 1.5 kb, 2 kb and 5 kb in length. In certain embodiments, the 5′ homology region comprises 1.1 kb and the 3′ homology region comprises 1 kb. The homology regions can be homologous to regions of the target gene and also, or instead, be homologous to regions upstream or downstream of the target gene. In one embodiment, the homology regions are homologous to chromosomal regions immediately adjacent to the target sequence. For example, in the case of the 5′ homology region, the homology region is homologous to a sequence having its most 3′ nucleotide immediately adjacent to the first (most 5′) nucleotide of the target sequence. In one embodiment, homology regions are homologous to chromosomal regions that are not immediately adjacent to the target sequence on the chromosome. In some embodiments, the 5′ and 3′ homologous regions are each 95-100% homologous to the cognate nucleic acid sequences flanking the target sequence.

To summarize the above-described component arrangement, the donor construct comprises, in order from 5′ to 3′, a 5′ homology region, a 5′ recombinase recognition site, a donor sequence, a 3′ recombinase recognition site, and a 3′ homology region. The donor construct can further include certain sequences that provide structural or functional support, such as sequences of a plasmid or other vector that supports propagation of the donor construct (e.g., pUC19 vector). The donor construct can, optionally, also include certain selectable markers or reporters, some of which may be flanked by recombinase recognition sites for subsequent activation, inactivation, or deletion. The recombinase recognition sites flanking the optional marker or reporter can be the same or different from the recombinase recognition sites flanking the donor sequence. In certain embodiments, a single type of donor construct is used to produce the conditional knock-out allele.

Concomitant with, or sequential to, introduction of the donor construct, a sequence-specific nuclease is introduced into the cell. The sequence-specific nuclease recognizes and binds to a specific sequence within the target gene and introduces a double-strand break in the target gene. As described above, the donor sequence is modified by at least one neutral mutation to reduce homologous recombination events that do not result in conditional knock-out alleles. In certain embodiments, the sequence-specific nuclease cleaves the target gene only once, i.e., a single double-strand break is introduced in the target gene during the methods described herein.

Examples of sequence-specific nucleases include zinc finger nucleases (ZFNs). ZFNs are recombinant proteins composed of DNA-binding zinc finger protein domains and effector nuclease domains. Zinc finger protein domains are ubiquitous protein domains, e.g., associated with transcription factors, that recognize and bind to specific DNA sequences. One of the “finger” domains can be composed of about thirty amino acids that include invariant histidine residues in complex with zinc. While over 10,000 zinc finger sequences have been identified thus far, the repertoire of zinc finger proteins has been further expanded by targeted amino acid substitutions in the zinc finger domains to create new zinc finger proteins designed to recognize a specific nucleotide sequence of interest. For example, phage display libraries have been used to screen zinc finger combinatorial libraries for desired sequence specificity (Rebar et al., Science 263:671-673 (1994); Jameson et al., Biochemistry 33:5689-5695 (1994); Choo et al., PNAS 91:11163-11167 (1994), each of which is incorporated herein as if set forth in its entirety). Zinc finger proteins with the desired sequence specificity can then be linked to an effector nuclease domain, e.g., as described in U.S. Pat. No. 6,824,978, such as FokI, described in PCT Application Publication Nos. WO1995/09233 and WO1994018313, each of which is incorporated herein by reference as if set forth in its entirety.

Another example of sequence-specific nucleases includes transcription activator-like effector endonucleases (TALEN), which comprise a TAL effector domain that binds to a specific nucleotide sequence and an endonuclease domain that catalyzes a double strand break at the target site. Examples of TALENs and methods of making and using are described by PCT Patent Application Publication No. WO2011072246, incorporated herein by reference as if set forth in its entirety.

Another example of a sequence-specific nuclease system that can be used with the methods and compositions described herein includes the Cas9/CRISPR system (Wiedenheft, B. et al. Nature 482,331-338 (2012); Jinek, M. et al. Science 337,816-821 (2012); Mali, P. et al. Science 339,823-826 (2013); Cong, L. et al. Science 339,819-823 (2013)). The Cas9/CRISPR (Clustered Regularly Interspaced Short Palindromic Repeats) system exploits RNA-guided DNA-binding and sequence-specific cleavage of target DNA. A guide RNA (gRNA) contains 20 nucleotides that are complementary to a target genomic DNA sequence upstream of a genomic PAM (protospacer adjacent motifs) site (NNG) and a constant RNA scaffold region. The Cas (CRISPR-associated) 9 protein binds to the gRNA and the target DNA to which the gRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, L. et al. Science 339, 819-823 (2013)). It is specifically contemplated that the inventive methods and compositions can be used with the single- or double-strand-inducing version of Cas9, as well as with other RNA-guided DNA nucleases, such as other bacterial Cas9-like systems. In some embodiments, the guide RNAs used in the methods described herein are those of SEQ ID NOS: 36-42, respectively, in order of appearance. The sequence-specific nuclease of the methods and compositions described herein can be engineered, chimeric, or isolated from an organism.

The sequence-specific nuclease can be introduced into the cell in form of a protein or in form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the donor construct can be delivered by any method appropriate for introducing nucleic acids into a cell.

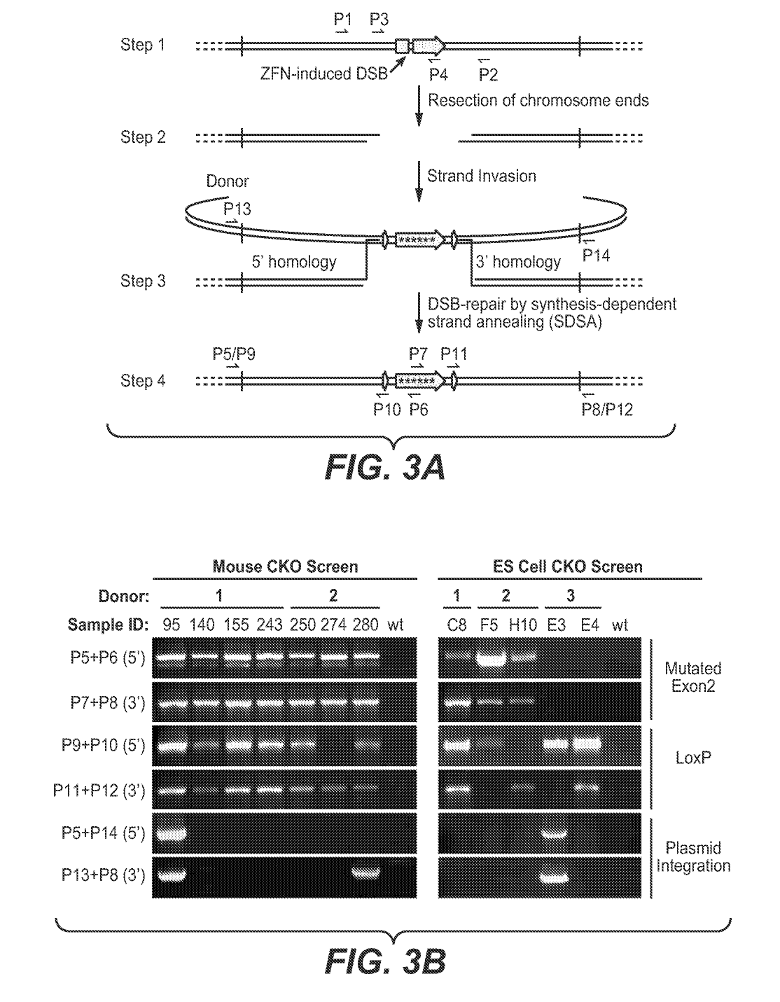

Without being limited by any particular mechanism or theory, following sequence-specific nuclease-introduced double-strand break in the target sequence (e.g., ZFN-induced DSB; FIG. 3A, Step 1), strand resection generates 3′single-stranded chromosome ends (FIG. 3A, Step 2). To initiate repair, the single-stranded chromosome ends anneal to complementary base pairs within the homology regions present on the donor construct by strand invasion (FIG. 3A, Step 3). The donor sequence can then be used as a template to extend the 3′ single-stranded ends by DNA polymerase-mediated strand extension. Following strand extension, the extended strand anneals to the single-stranded chromosome end on the other side of the original double-strand break and repair is completed by DNA synthesis, using the extended strand as template, and ligation. The resulting double-stranded DNA contains the donor sequence flanked by recombinase recognition sites (FIG. 3A, Step 4).

This synthesis-dependent strand annealing model of double-strand break repair is consistent with the observation that very large stretches of foreign DNA with little or no homology to endogenous sequence, such as a reporter gene, can be inserted precisely into the point of the double-strand break. Consequently, donor sequences flanked by recombinase recognition sites can be integrated at the double strand break by resection of the free chromosome ends to expose regions around the target sequence that are substantially homologous to the homology regions on the donor construct (FIG. 3A). The homology regions can be of any length suitable for placement in a donor construct and effective in mediating strand annealing as described above, e.g., a combined length of 10-5000 bp, 100-1000 bp, 500-600 bp, or 537 bp. These steps, thus, create a conditional knock-out allele at the site of the target gene, i.e., an allele comprising the donor sequence flanked by the recombinase recognition sites that produces a phenotype that is substantially similar to, or indistinguishable from, that produced by the cognate target gene allele. Two phenotypes are substantially similar or indistinguishable if upon standard inspection by a skilled artisan the nature of the underlying allele of the target gene cannot be detected. In some embodiments, the methods described herein produce cells carrying heterozygous conditional knock-out alleles or homozygous conditional knock-out alleles, i.e., less than all or all of the endogenous alleles are replaced by the conditional knock-out allele.

The target gene can be any nucleic acid molecule encoding a protein (or fragments thereof) within the genetic material of the cell that is being targeted by the donor construct to produce a conditional knock-out version of the gene. For example, a target gene can be a gene located on the chromosome of a eukaryotic cell that encodes a protein of unknown function or that is involved in a cellular process. Such gene can be composed of a series of exons and introns. A target sequence can include exon, intron (including artificial intron), or regulatory sequences of the target gene, or various combinations thereof. A target sequence can include the entire target gene.

The cell can be any eukaryotic cell, e.g., an isolated cell of an animal, such as a totipotent, pluripotent, or adult stem cell, a zygote, or a somatic cell. In certain embodiments, cells for use in the methods described herein are cells of non-human animals, such as domesticated animals (e.g., cows, sheep, cats, dogs, and horses), primates (e.g., non-human primates such as monkeys), rabbits, fish, rodents (e.g., mice, rats, hamsters, guinea pigs), flies, and worms. In certain embodiments, cells for use in the methods are human cells. The methods and compositions described herein can be used to target any genomic locus. Several specific examples of targeting different loci are described herein. In certain embodiments, the methods and compositions described herein can be used to target more than one genomic locus within a cell, i.e., for multiplex gene targeting.

In a further particular aspect of the invention, a conditional knock-out animal is produced using the methods described herein. To produce a conditional knock-out animal, a donor construct and a sequence-specific nuclease are introduced into a cell, such as a zygote or a pluripotent stem cell, such as an embryonic stem cell or an induced pluripotent stem cell, or an adult stem cell, to create at least one conditional knock-out allele in the cell. Methods for screening for the desired genotype are well known in the art and include PCR analysis, e.g., as described herein in the specific examples. The cell is then introduced into a female carrier animal to produce the conditional knock-out animal from the cell, for example as disclosed by U.S. Pat. No. 7,13,608, incorporated herein by reference as if set forth in its entirety. In certain embodiments, the cell is expanded to a two-cell stage, introduced into a blastocyst, or otherwise cultured or associated with additional cells prior to introduction into the carrier animal. In certain embodiments, the resulting conditional knock-out animal carries the conditional knock-out allele in its germline such that the conditional knock-out allele can be passed on to future generations.

In a further particular aspect of the invention, the methods and compositions described herein can be used to produce a knock-out allele. This method includes excising, inverting, or otherwise inhibiting normal expression of the recombinase recognition site-flanked donor sequence, once incorporated into the genome as conditional knock-out allele. The conditional knock-out allele is converted to a knock-out allele by introducing a recombinase into the cell that specifically recognizes the recombinase recognition sites. E.g., Araki et al., Proc. Natl. Acad. Sci. USA 92:160-164 (1995). The recombinase is an enzyme that recognizes specific polynucleotide sequences (recombinase recognition sites) that flank an intervening polynucleotide and catalyzes a reciprocal strand exchange, resulting in inversion or excision of the intervening polynucleotide. One of skill in the art recognizes the advantageous efficiency of selecting for use in the methods described herein a recombinase that specifically recognizes the recombinase recognition sites within the donor construct.

The recombinase can be introduced into the cell containing the donor construct by any method in form of a protein or nucleotide sequence encoding the recombinase protein. To produce a knock-out animal, the conditional knock-out animal, produced as described above, is crossed to a transgenic animal having a transgene encoding a recombinase protein that catalyzes recombination at the 5′ and 3′ recombinase recognition site. Examples of animals carrying a recombinase transgene are known in the art and disclosed, for example, by U.S. Pat. No. 7,135,608, incorporated herein by reference as if set forth in its entirety. In some embodiments, the transgene encoding the recombinase is under the control of a tissue-specific promoter, such that the recombinase is expressed and, consequently, the knock-out allele is produced, only in such tissue. In some embodiments, the transgene encoding the recombinase is under the control of an inducible promoter, such that recombinase expression can be induced at a specific time. For example, the activation of Tet-On or Tet-Off promoters can be controlled by tetracycline or one of its derivatives. In some embodiments, the recombinase-encoding transgene is expressed only at a certain stage of development or in response to a compound administered to the animal. Examples of recombinases suitable for use in the methods disclosed herein include any version of P1 Cre recombinase, any version of FLP recombinase (flippase), and any version of Dre recombinase, including any inducible version of these recombinases (e.g., fusions to a hormone-responsive domain such as CreERT2 and Cre-PR, or tetracycline-regulated recombinase).

B. Exemplary Compositions

In a further specific aspect of the invention, a composition for generating a conditional knock-out allele of a target gene is provided. Such composition includes a donor construct comprising a 5′ homology region, a 5′ recombinase recognition site, a donor sequence, a 3′ recombinase recognition site, and a 3′ homology region, as described herein. The donor sequence comprises a target sequence having at least one neutral mutation, as described herein. The composition further comprises a sequence-specific nuclease that recognizes the target gene.

In certain embodiments, the sequence-specific nuclease is a zinc finger nuclease or a transcription activator-like effector nuclease. In certain embodiments, the recombinase recognition site is a loxP site or an frt site. Optionally, the composition can also include a recombinase, as described herein.

In a further aspect of the invention, a donor construct comprising the sequence shown in FIG. 4A (SEQ ID NO: 30), FIG. 4B (SEQ ID NO: 31), or FIG. 14C (SEQ ID NOS: 44-46).

In a further aspect of the invention, a guide RNA comprising the sequence shown in FIG. 14A (SEQ ID NOS: 36-42) is provided.

In a further aspect of the invention, a cell comprising the donor construct comprising the sequence shown in FIG. 4A (SEQ ID NO: 30), FIG. 4B (SEQ ID NO: 31), or FIG. 14C (SEQ ID NOS: 44-46) is provided. This cell may be isolated from an animal produced by the methods described herein.

The invention can be further understood by reference to the following non-limiting examples of certain embodiments of the invention.

### III. Examples

The following are examples of methods and compositions of the invention. It is understood that various other embodiments may be practiced, given the general description provided above.

### Example 1

### Pronuclear Microinjection of Lrp5 ZFN mRNA into C57BL/6N Fertilized Eggs

A custom eHi-Fi CompoZr® ZFN pair targeting exon 2 of mouse Low-density lipoprotein receptor-related protein 5 (Lrp5) was obtained from Sigma-Aldrich. The ZFNs harbor an optimized (eHi-Fi) FokI endonuclease interface that significantly increases its efficiency in introducing double-strand breaks (Doyon, Y. et al. Nat Meth 8, 74-79 (2011)) at

(SEQ ID NO: 29)
5′-gacttccagttctccaagggtgctgtgtactggacagat-3′

(ZFN cleavage site is underlined). No significant potential off-site target activity was observed. Messenger RNA (mRNA) encoding the ZFN pair was stored at −80° C. prior to use. mRNA (Sigma-Aldrich) was used for pronuclear microinjection and the two plasmids encoding the ZFN pair were used for ES cell electroporation.

To determine endonuclease activity, various concentrations of mRNAs encoding the Lrp5 ZFNs were microinjected into the pronucleus of C57BL/6N zygotes (Table 1). Lrp5 ZFN mRNA (2 μg of each ZFN in 5 μl) was thawed and diluted to 50 ng/μl in RNase- and DNase-free microinjection buffer (10 mM Tris and 1 mM of EDTA, PH 8.0). ZFN microinjections, Lrp5 ZFN mRNA was diluted to working concentrations of 2, 3, 4, or 5 ng/μl. Mouse zygotes were obtained from superovulated C57BL/6N females mated to C57BL/6N males (Charles River) the day before microinjection. Zygotes were harvested with M2 medium and microinjected in M2 following standard procedures (Nagy, A., et al., Manipulating the Mouse Embryo: A Laboratory Manual, Third Edition (Cold Spring Harbor Laboratory Press, Cold Spring Harbor, USA, (2002)) and transferred into oviducts of E0.5 pseudopregnant ICR females (Taconic), 30 embryos per pseudopregnant female. ICR females were fed a 9% high fat diet (Harlan, catalog #2019) after embryo transfer surgery until the pups were weaned.

TABLE 1
Pronuclear microinjection of Lrp5 ZFN mRNA
into C57BL/6N fertilized eggs. KO mutants include
mice with one or more mutant alleles.

| Micro- | | Zygotes | | | | |
|---|---|---|---|---|---|---|
| injection | mRNA | transferred | | birth | | KO rate |
| Exper- | conc. | after | Pups | rate | | % (KO/ |
| iment | (ng/μl) | injection | born | % | KOs | born) |
| 1 | 2 | 168 | 48 | 29 | 20 | 42 |
| 2 | 3 | 114 | 15 | 13 | 2 | 13 |
| 3 | 3 | 150 | 39 | 26 | 15 | 38 |
| 4 | 4 | 108 | 21 | 19 | 8 | 38 |
| 5 | 5 | 174 | 57 | 33 | 36 | 63 |

KO = knock-out.

DNA from the resulting pups was isolated from tail tissue and analyzed by PCR amplification and subsequent sequencing to identify large and small mutations. Genomic tail DNA was purified using Extract-N-Amp Tissue PCR kit (Sigma, Cat# XNAT2) or using Qiagen DNeasy 96 Blood and Tissue kit (Qiagen Cat#69582). To determine ZFN-mediated mutation efficiency and to characterize the types of mutations caused by NHEJ repair, a 3-step PCR approach was performed. In the first step, an outer PCR using primers P1 and P2 was performed to detect large deletions or insertions. In the second step, an inner PCR using primers P3 and P4 was performed to detect small to medium size deletions or insertions. In the third step, direct sequencing of the inner PCR reaction product using primers P3 and P4 was performed to identify 1 to 20 base pair changes. Individual chromatograms were analyzed using Sequencher 4.10.1 (Gene Codes Corp.). If two distinct traces were detected, base pair calls for each individual allele were determined manually. Alleles from a subset of mutants were further analyzed by PCR TOPO subcloning (Invitrogen, Cat# K4575-J10). Twelve to twenty-four TOPO clones per mouse were sequenced using M13F and M13R primers.

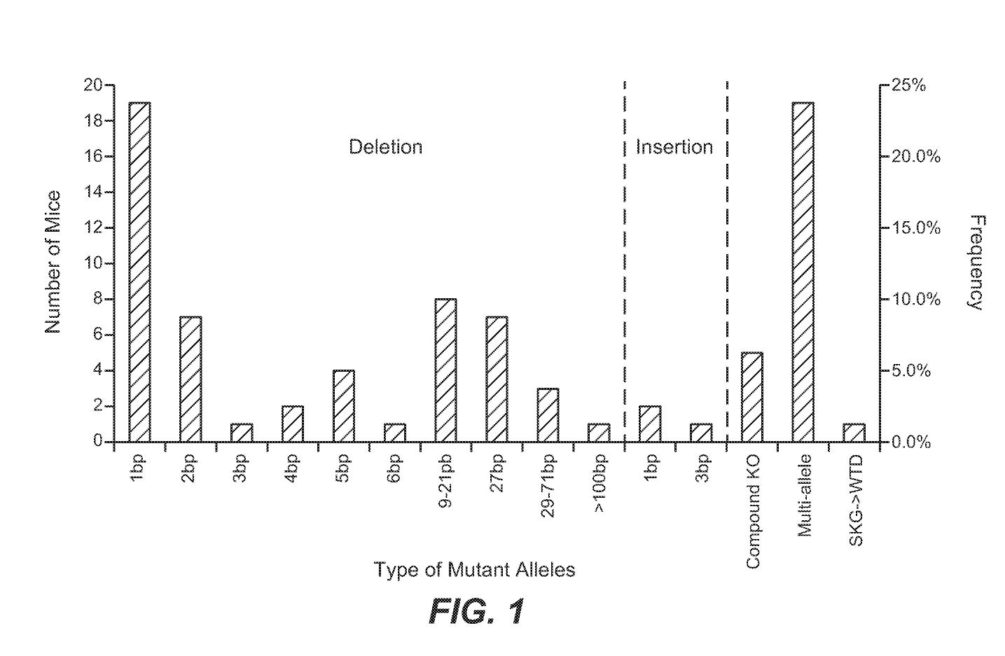

Mutation rates of up to 63% of live-born pups were observed (5 ng/μl ZFN mRNA). The mutations ranged widely from insertions of one to three by and deletions ranging from a single by up to ˜100 bp as well as one large ˜800 bp deletion (summarized in FIG. 1). Multiple chimeric animals carrying more than two alleles were identified, likely resulting from continuing ZFN activity after the first cell division. Furthermore, five animals were compound mutants, i.e., these animals carried two independent mutant alleles of the same gene and no detectable wildtype allele of the gene, indicating ZFN activity on both chromosomes at the one cell stage.

### Example 2

### Direct Generation of Functional Homozygous Mutant Alleles by Microinjection of Sequence-Specific Endonucleases

LRP5 plays an obligatory role in retinal vascular development by serving as a co-receptor for NORRIN. Disrupted NORRIN signaling leads to vascular defects characterized by a failure to form capillary beds in the deeper layers of the retina, as well as vascular leakage (Xia, C.-H. et al., Human Molecular Genetics 17, 1605-1612 (2008); Xia, C.-H., PLoS ONE 5, e11676 (2010); Junge, H. J. et al., Cell 139, 299-311 (2009)). Thus, 2-month-old mice with compound in-frame and out-of-frame deletions in Lrp5 were generating as described in Example 1 and were examined for retinal vascular development. Animal #542 is a chimeric functional heterozygous that served as control. This animal carries one wild-type allele (a small 3 bp in-frame deletion appeared to be silent) and an allele with a 1 bp out-of-frame deletion. Animal #495 contains a 4 bp out-of-frame deletion allele and a 1 bp out-of-frame deletion allele. Animal #519 contains a 29 bp out-of-frame deletion allele and a 17 bp out-of-frame deletion allele. Animal #555 has a 3 bp in-frame deletion allele and a 1 bp out-of-frame deletion allele and is a functional heterozygote.

For phenotypic analysis, animals carrying Lrp5 mutations were analyzed by fluorescein angiography. Mice were anesthetized with a mixture of ketamine/xylazine (80 mg/kg; 7.5 mg/kg) and dilating the eyes with 1% Tropicamide (Akorn, Inc.). Fluorescein angiography was performed after intraperitoneal injection of sterile 10% fluorescein solution (100 μl, AK-Fluor; Akorn, Inc.). Images were captured 1 minute after fluorescein injection using imaging setting of 0 focus and 50 sensitivity.

For histologic analysis, mice were sacrificed two days after angiography, enucleated, and processed for histology. Eyes were fixed in 4% paraformaldehyde (PFA) prior to dissection of retinas for whole mount histology, or cryoprotected in 30% sucrose overnight and embedded in Tissue-Tek® OCT Compound (Sakura) for frozen sections. Isolectin-B4 staining of whole mounts and sections was performed as previously described (Gerhardt, H. et al., J. Cell. Biol. 161, 1163-1177 (2003)). For frozen sectioning, cornea and lens were removed and eyes were washed extensively in PBS to remove residual PFA. Frozen 12 μm sections were prepared and stained for MECA32, an antigen of the fenestrated endothelial cell marker PLVAP, essentially as described by Junge et al, Cell 139, 299-311 (2009).

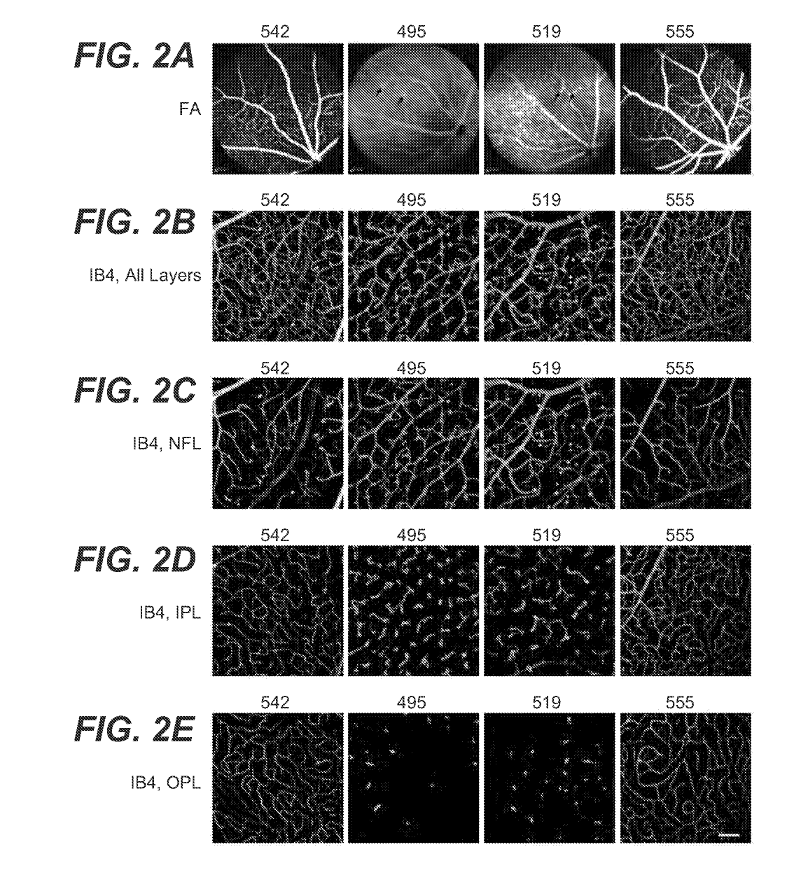

The retinal phenotype of three compound mutant mice (495, 519 and 555) and a control heterozygous mutant mouse carrying one wildtype allele (542) is shown in FIG. 2. Mice carrying the compound mutations displayed an Lrp5 null phenotype. Fluorescent angiography revealed that mice 542 and 555 display no apparent neovascular defects or vessel leakage (FIG. 2A). In contrast, mice 495 and 519, which contain compound out-of-frame deletions in both alleles of Lrp5, displayed numerous precapillary arteriole occlusions (FIG. 2A, arrows pointing to examples of precapillary arteriole occlusions) and significant vascular leakage, as indicated by the diffuse fluorescein signal throughout the retina. Scale bar in the bottom right panel of FIG. 2 represents 200 μm for all panels in FIG. 2A.

Confocal projections of isolectin-stained wholemount retinas confirmed the Lrp5 null phenotype of compound mutant mice 495 and 519. For each mouse, a projection of the maximum depth of the retina containing all three vascular layers (FIG. 2B), and images derived from projection of a single vascular layer residing in the nerve fiber layer (NFL, FIG. 2C), inner plexiform layer (IPL, FIG. 2D), and outer plexiform layer (OPL, FIG. 2E) were analyzed. While functional heterozygous retinas (542 and 555) contain a dense, well-organized three-tiered network of vessels, compound knock-out retinas (495 and 519) have irregular vasculature with reduced density (FIG. 2B, C). In addition, 542 and 555 contain normal capillary networks in the IPL (FIG. 2D) and OPL (FIG. 2E), whereas compound KO mice (495 and 555) have abnormal neovascular clusters in the IPL (FIG. 2D) and a small number of endothelial cell clusters in the OPL (FIG. 2E). Scale bar in the bottom right panel of FIG. 2 represents 100 μm for all panels in FIG. 2B-E.

In summary, mutant 555, carrying a loss of function allele with a 1 bp deletion and a functional allele with 3 bp in-frame deletion, displayed a normal retinal phenotype, while mutant 495, carrying a 4 bp and a 1 bp deletion, and mutant 519, carrying two larger deletions (17 and 29 bp), were phenotypically homozygous null, with a phenotype recapitulating what has been reported previously (Xia, C.-H. et al., Human Molecular Genetics 17, 1605-1612 (2008)). These results demonstrate that microinjection of sequence-specific endonucleases can produce functional homozygotes (compound mutants) directly, although it is not known if these animals are compound mutants in all cells.

### Example 3

### Generation of Conditional Knock-Out Alleles by Co-Microinjection of Lrp5 Exon2 ZFN mRNA and Donor Construct

FIG. 3A depicts a schematic outline of the strategy employed to generate a conditional knock-out allele (Gu, H., Science 265, 103-106 (1994)) of Lrp5, targeting exon 2. The ZFN pair introduces a double-strand break in Lrp5 exon 2 (indicated by interrupted block arrow). The break is repaired by invasion of the donor plasmid through strand invasion and homologous recombination between the 5′ and 3′ Lrp5 homology regions of the donor plasmid and the respective homologous sequences 5′ and 3′ of exon 2. The resulting locus contains the codon-optimized Lrp5 exon 2 flanked by two loxP sites (FIG. 1A, bottom).

The 5′ and 3′ Lrp5 homology regions in the donor plasmid were 1.1 and 1 kb, respectively, in length. Codon-modified (donor 1, FIG. 4A) and wildtype (donor 3, FIG. 4C) donor sequences were synthesized by Blue Heron/Origene (Bothell, Wash.) into a modified pUC19 vector. Donor 2 (FIG. 4B) was generated from donor 3 by replacing a 300 bp MscI-BamHI fragment with a synthesized fragment containing seven silent mutations to abrogate ZFN recognition. The insert in donor 1 is in opposite orientation compared to the insert in donors 2 and 3. Therefore, PCR amplification using primers that bind the plasmid backbone in combination with Lrp5 locus-specific primers was conducted using primer combinations of the opposite orientation. The donor sequence, with the exception of the loxP sites, corresponds to mouse genome assembly NCBI37/mm9 chr.19:3658179-3660815. Circular donor plasmids were used in all experiments.

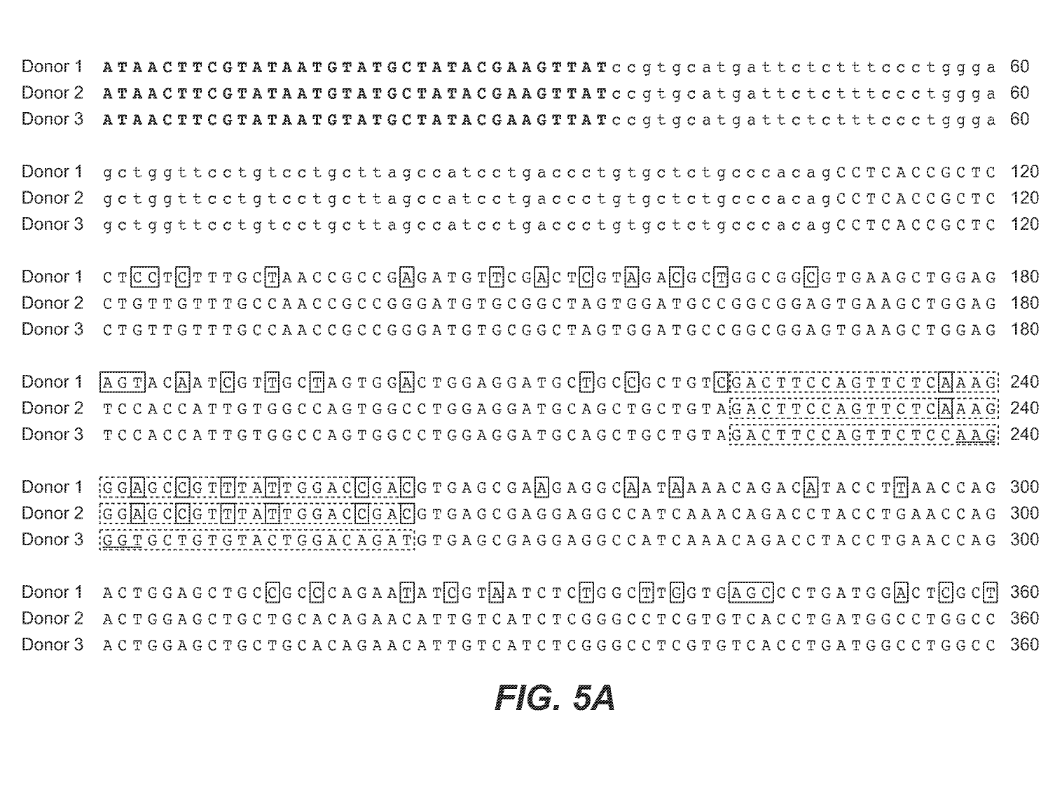

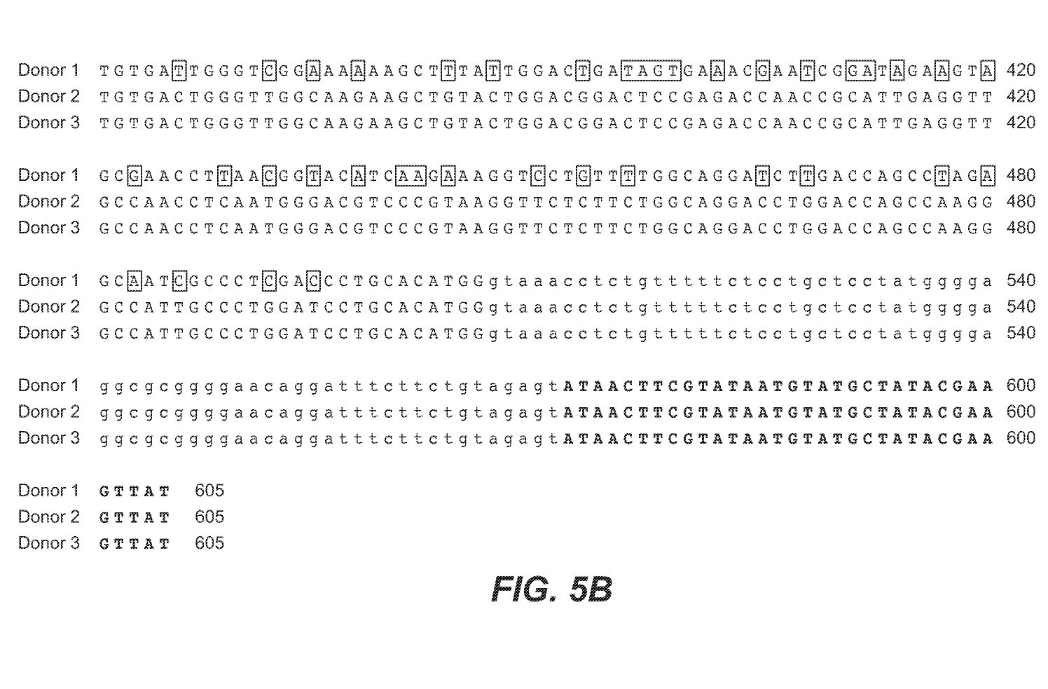

Silent mutations were introduced into the wildtype Lrp5 exon 2 sequence to produce a codon-optimized version maintaining the protein-coding potential of the exon, but reducing the overall homology between wildtype C57BL/6 and donor Lrp5 exon 2 to only 78% (donor 1, FIG. 4A; FIG. 5). To preserve normal RNA splicing, the first 13 bp or the last 11 bp of exon 2 were excluded from modification. FIG. 5 depicts a sequence alignment of the three Lrp5 conditional knock-out DNA donors, excluding the 1.1 kb 5′ homology and 1 kb 3′ homology regions. Alignment was done using the alignment program ClustalW2, available at http://www.ebi.ac.uk/Tools/msa/clustalw2/. The overall homology between donor 1 (codon modified) and donor 3 (wildtype) exon 2 is 311/397=78%. The overall homology between donor 2 (ZFN binding site-modified only) and donor 3 exon 2 is 390/397=98%. LoxP sites are indicated by uppercase bold letters, intron sequences are indicated by lowercase letters, and the exon 2 (wild type or modified) sequences are indicated by uppercase letters. The ZFN binding sites are boxed with dashed lines and the sequence at which the wild type exon 2 is cleaved is underlined. Silent mutations are boxed with solid lines.

Different combinations of ZFN mRNA and donor constructs were co-microinjected into C57BL/6N pronuclei (Table 2), essentially as described in Experiment 1, except that ZFN mRNA and donor construct were diluted together to working concentration (2.5-5 ng/μl for ZFN mRNA and 2.5 or 3 ng/μl for donor construct).

TABLE 2
Co-microinjection of Lrp5 ZFN mRNA (mRNA) and CKO donor 1 plasmid.
KO mutants include mice with one or more mutant alleles.

| Co-micro- | mRNA + | Zygotes | | | | | | |
|---|---|---|---|---|---|---|---|---|
| injection | DNA | transferred | | birth | | KO rate | | CKO rate |
| Exper- | conc. | after | Pups | rate | | % (KO/ | | % (CKO/ |
| iment | (ng/μl) | injection | born | % | KOs | born) | CKOs | born) |
| 1 | 2.5 + 2.5 | 120 | 28 | 23 | 11 | 39 | 0 | — |
| 2 | 2.5 + 2.5 | 128 | 35 | 27 | 10 | 29 | 0 | — |
| 3 | 2.5 + 3 | 114 | 6 | 5 | 4 | 67 | 0 | — |
| 4 | 3 + 2.5 | 121 | 10 | 8 | 6 | 60 | 0 | — |
| 5 | 3 + 3 | 126 | 23 | 18 | 7 | 30 | 0^{a} | — |
| 6 | 4.5 + 2.5 | 118 | 18 | 15 | 5 | 28 | 0 | — |
| 7 | 4.5 + 3 | 146 | 30 | 21 | 13 | 43 | 2^{b} | 6.7 |
| 8 | 5 + 2.5 | 102 | 18 | 18 | 8 | 44 | 0 | — |

KO = knock-out; CKO = conditional knock-out.
^{a}One mouse (#95) was a false positive (donor 1 plasmid integrated into Lrp5 locus).
^{b}Mice #140 and #155.

DNA isolated from tail samples from the 168 resulting pups were analyzed to identify mice that carry a conditional knock-out allele (FIG. 3B). The respective primer pairs used for analysis of mutants in the absence (P1-P4) or presence (P5-P12) of donor plasmid are indicated in FIG. 3B. First, the overall ZFN mutation frequency was determined as described in Experiment 1 and 2. Initial screening to identify mice carrying a potential conditional knock-out allele was performed by assaying for presence of the 5′ LoxP site using a 5′ nuclease assay (TaqMan®, Livak, K. J., Genet. Anal. 14, 143-149 (1999)). In brief, 20 μl reactions were constructed with a 2× Qiagen Type-it Fast SNP Probe PCR master mix, 50-120 ng template DNA, 400 nM primers and 200 nM fluorogenic Locked Nucleic Acid (LNA)-based probe specific for LoxP site recognition (Weis, B., BMC Biotechnol 10, 75 (2010)). Reactions were thermally cycled in an Applied Biosystems 7900HT (Life Technologies). Presence of the 5′ LoxP was determined by analysis with Applied Biosystems Sequence Detection Software, version 2.3 (Life Technologies), by visualization of fluorescence evolution in the multi-component and amplification plots. Lrp5 Locus-specific PCR analysis using primers P5/P6 was then performed to detect a 5′ product specific for the codon-modified Lrp5 exon 2 sequence present on both donor 1 and 2 (but not donor 3 used for the ES cell experiment of Example 4). Similarly, PCR using primers P7/P8 was performed to analyze the 3′ end. To validate the presence of both 5′ and 3′ loxP, PCR analysis using primers P9/P10 and P11/P12, respectively, was performed which will result in products only if the appropriate loxP sequence is present in the Lrp5 locus. As the DNA was isolated from a mixture of chimeric subclones, false positive results were observed, i.e., PCR products appear to be positive for 5′-3′-floxed Lrp5 alleles even in the absence of such true conditional knock-out alleles. False positive results could be produced, for example, if one allele carries only the 5′ loxP site and another allele carries only the 3′ loxP site. To confirm the presence of a conditional knock-out allele, as opposed to false positive, a ˜2.8 kb Lrp5 exon 2 PCR product was amplified using primers P5/P8 (both primers anneal outside the donor homology arms), cloned using and TOPO cloning (Life Technologies), and fully sequenced. This analysis identified conditional knock-out alleles, alleles with only a single loxP site, and alleles with donor-derived exon 2 sequence only (i.e., no loxP sites). Alleles identified as false positives by sequencing analysis were analyzed for the presence of an integrated copy of the entire donor vector in the Lrp5 allele by additional PCR using flanking primers P5 and P8 in combination with donor plasmid backbone-specific primers. Presence of random genomic insertions was determined with primers P6 and P7 (donor 1 and donor 2) in combination with donor plasmid backbone-specific primers (P13-P14). For random insertions of donor 3, donor plasmid backbone-specific primers (P13-P14) were used in combination with primers P15 and P16 that bind to the wildtype Lrp5 sequence of donor 3. All primer sequences and reaction conditions are set forth in Table 3. The conditions for all PCR studies are set forth in Table 7.

Two mice (#140 and #155) were confirmed as carrying conditional knock-out alleles by full sequencing of a cloned PCR product obtained using primers located outside of the homology regions. For both mice, the conditional knock-out allele was transmitted to their progeny. In addition to the conditional knock-out allele, animal #155 also had one low frequency allele (not transmitted to progeny) with the 5′ loxP site only. Animal #95 was a false positive as initial PCR analysis suggested a conditional knock-out allele, but detailed analysis revealed that a full-length donor plasmid was instead integrated into Lrp5 exon 2. The knock-out mutation rates for each combination of ZFN mRNA and donor DNA ranged from 28 to 67% (Table 2).

| Primer | | |
|---|---|---|
| Number | Primer Sequence (5′−>3′) | Purpose |
| 1 | CATGTGCCTTTGAAGAGCACACC | To detect large deletions/insertions |
| | (SEQ ID NO: 1) | |
| 2 | ACTCCACGGTCCTGGGATTATAGA | To detect large deletions/insertions |
| | (SEQ ID NO: 2) | |
| 3 | GGCCTATCACTAAGGGAGCC | To detect small to medium |
| | (SEQ ID NO: 3) | deletions/insertions |
| 4 | GCCCGAGATGACAATGTTCT | To detect small to medium |
| | (SEQ ID NO: 4) | deletions/insertions |
| 5 | CGAGCTTTTCTTAGTGATCTTTTAAG | 5′ flanking primer (outside of |
| | (SEQ ID NO: 5) | homology arm) |
| 6 | CTCACGTCGGTCCAATAAACG | To detect donor 1 and donor 2 |
| | (SEQ ID NO: 6) | exon2 sequence |
| 7 | CGTTTATTGGACCGACGTGAG | To detect donor a and donor 2 |
| | (SEQ ID NO: 7) | exon2 sequence |
| 8 | CCTAGACTGCAGTGAAGGACAT | 3′ flanking primer (outside of |
| | (SEQ ID NO: 8) | homology arm) |
| 9 | GCTCACGAGCTTTTCTTAGTGATCTTTTAAGG | 5′ flanking primer (outside of |
| | (SEQ ID NO: 9) | homology arm) |
| 10 | GAGAATCATGCACGGATAACTTCGTATAGC | To detect 5′ loxP integration |
| | (SEQ ID NO: 10) | |
| 11 | CAGGATTTCTTCTGTAGAGTATAACTTCGTATAATG | To detect 3′ loxP integration |
| | (SEQ ID NO: 11) | |
| 12 | CCTAGACTGCAGTGAAGGACATTCAC | 3′ flanking primer (outside of |
| | (SEQ ID NO: 12) | homology arm) |
| 13 | GGATAACAATTTCACACAGGAAACAGCTA | To detect random insertions or |
| | (SEQ ID NO: 13) | plasmid integration |
| 14 | GTAAAACGACGGCCAGTGAATTGG | To detect random insertions or |
| | (SEQ ID NO: 14) | plasmid integration |
| 15 | CAGGGAAAGAGAATCATGCAC | To detect donor 3 random |
| | (SEQ ID NO: 15) | insertions |
| 16 | CTGCACATGGGTAAACCTCTG | To detect donor 3 random |
| | (SEQ ID NO: 16) | insertions |
| 17 | CACCTGAACTACTGAAAG | To detect 5′ loxP |
| | (SEQ ID NO: 17) | |
| 18 | CAGGGAAAGAGAATCATG | To detect 5′ loxP |
| | (SEQ ID NO: 18) | |
| 19 | F-ATAACTTCG-IQ-TATAGCATACATTATAC-Q | To detect 5′ loxP |
| | (SEQ ID NO: 19) | |

Table 3. Primer nucleotide sequences. Primer P19: F = fluorophore (fluorescein);
Q = quencher (Iowa Black FQ, Integrated DNA Technologies); IQ = internal quencher
(ZEN, Integrated DNA Technologies). LNA bp are underlined.

The co-injection experiment in mouse zygotes (4.5 ng/μl ZFN mRNA and 3 ng/μl donor DNA) were repeated, by co-injecting Lrp5 ZFN mRNA and either donor 1 or a floxed codon-optimized exon 2 donor that carries seven silent mutations with respect to the wildtype sequence, which abrogates ZFN-binding and cleaving of the donor (donor 2, FIG. 4B; FIG. 5). The results of these experiments are summarized in Table 4. Co-injection of donor 1 with Lrp5 ZFN mRNA resulted in one out of twelve pups that carried a conditional knock-out allele (#243, 8.3% conditional knock-out rate). Co-injection of donor 2 with Lrp5 ZFN mRNA resulted in three out of thirty-five pups (8.6%) that carried donor 2 exon sequence in the Lrp5 locus. However, only one of these was subsequently confirmed as carrying a low frequency conditional knock-out allele (#250). The second of the three animals carried an allele with the 3′ loxP site only (#274); the last animal (#280) harbored one allele with donor 2 exon sequence only (no loxP sites) and another allele with fully integrated donor 2 plasmid (false positive). These results suggest that the donor plasmid with the lowest sequence homology to the endogenous Lrp5 exon 2 sequence (donor 1, FIG. 4A) was more efficient at generating conditional knock-out alleles.

TABLE 4
Co-microinjection of Lrp5 ZFN mRNA and CKO donor 1 or donor
2 plasmids. All experiments were performed using 4.5 ng/μl ZFN
mRNA and 3 ng/μl donor plasmid DNA. Overall CKO rate was
1/12 (8.3%) for donor 1 and 1/35 (2.9%) for donor 2.

| Co-micro- | | Zygotes | | | | | | |
|---|---|---|---|---|---|---|---|---|
| injection | | transferred | | birth | | KO rate | | CKO rate |
| Exper- | | after | Pups | rate | | % (KO/ | | % (CKO/ |
| iment | Plasmid | injection | born | % | KOs | born) | CKOs | born) |
| 1 | Donor 1 | 50 | 10 | 20 | 10 | 100 | 1^{a} | 10 |
| 2 | Donor 1 | 67 | 2 | 3 | 1 | 50 | 0 | — |
| 3 | Donor 1 | 70 | 0 | — | — | — | — | — |
| 1 | Donor 2 | 42 | 11 | 26 | 4 | 36 | 1^{b} | 9 |
| 2 | Donor 2 | 73 | 15 | 21 | 10 | 67 | 0^{c} | — |
| 3 | Donor 2 | 78 | 9 | 11.5 | 7 | 78 | 0^{d} | — |

^{a}Mouse #243;
^{b}Mouse #250;
^{c}One mouse (#274) carried a 3′loxP site only allele;
^{d}One mouse (#280) carried one allele with donor 2 exon only (no loxP sites) and one false positive allele (donor 2 plasmid integrated into Lrp5 locus).

### Example 4

### Generation of Conditional Knock-Out Alleles by Co-Electroporation of Lrp5 Exon2 ZFN and Donor Plasmid

C57BL/6N ES cells were co-transfected by electroporation with plasmids encoding the two Lrp5 ZFN pair components alone, or along with either donor plasmid used for the microinjection experiments, or with an unmodified floxed wildtype Lrp5 exon 2 plasmid (donor 3). C2 ES cells (Gertsenstein, M. et al., PLoS ONE 5, e11260 (2010)) were cultured, expanded, and electroporated using established methods (Nagy, A., Gertsenstein, M., Vintersten, K. and Behringer, R. Manipulating the Mouse Embryo: A Laboratory Manual, Third Edition. 800 (Cold Spring Harbor Laboratory Press: 2002)). In brief, fifteen million cells were electroporated with 15 μg of each ZFN plasmid with or without 15 μg donor plasmid. Electroporated cells were recovered in media and serial dilutions were plated on 10 cm plates on a feeder layer. Cells were grown for 7-8 days after which 144 clones (1.5 96 well plate) from each experiment were picked and placed into 96-well plates with feeder cells for expansion. Two days after plating, the cells were split 1:2 into new 96-well plates with feeder cells. One plate was then stored at −80° C. and the other plate was split into a new 96-well plate with 1% gelatin only without feeders cells, for DNA analysis. DNA was isolated as described in Example 1 except that ES cells were lysed over-night and DNA was precipitated, washed, and resuspended in TE buffer the following day, essentially as described by Ramirez-Solis, R. et al., Anal Biochem 201, 331-335 (1992).

TABLE 5
Electroporation of plasmids encoding the Lrp5 ZFN pair
alone or in combination with CKO donors 1, 2, or 3 into
C57BL/6N ES cells. All experiments were performed using
15 μg donor DNA and/or 15 μg each of ZFN1 and ZFN2.

| ES cell | | | | KO rate | | CKO rate |
|---|---|---|---|---|---|---|
| exper- | | Colonies | | % (KO/ | | % (CKO/ |
| iment | Plasmid | screened | KOs | analyzed) | CKOs | screened) |
| 1 | None | 144 | 24 | 17 | NA | NA |
| 2 | Donor 1 | 144 | ND | ND | 1^{a} | 0.7% |
| 3 | Donor 2 | 144 | ND | ND | 0^{b} | — |
| 2 | Donor 3 | 144 | ND | ND | 1^{c} | 0.7% |

^{a}Donor 1 ES clone #C8;
^{b}one donor 2 clone (F5) carried a 5′ loxP only allele and a donor 2 exon only allele (no loxP sites), clone H10 carried a 3′ loxP only allele;
^{c}two donor 3 clones (E3 and E4) carried alleles with 5′ loxP only. Clone E3 also carried a false positive allele (donor 3 plasmid integration). Clone E4 also carried a true CKO minor allele (one positive out of 240 TOPO clones sequenced).
ND: not investigated; NA: not applicable.

Results of the DNA analysis are shown in FIG. 3B, right, and the results are summarized in Table 5. The overall frequency of knock-out alleles observed in ES cells using electroporation (17%) was lower than obtained in vivo via pronuclear injection. The genetic alteration patterns from the ES cell electroporation experiment were similar to those observed after microinjection. Co-electroporation of donor 1 with Lrp5 ZFN plasmid resulted in one conditional knock-out clone (clone C8) out of 144 analyzed. Co-electroporation of donor 2 with Lrp5 ZFN plasmid resulted in two ES cell clones out of 144 analyzed that carry alleles derived from the donor. One of these clones (H10) carried the 3′ loxP site allele only; the other (F5) carried one allele with donor 2 sequence only (no loxP sites) and one allele with the 5′ loxP site only. Co-electroporation of donor 3 (wildtype) with Lrp5 ZFN mRNA resulted in two targeted ES cell clones (E3 and E4). Both contained one allele with the 5′ loxP site only. In addition, E3 carried another allele resulting from integration of donor 3 plasmid (false positive). Interestingly, clone E4 also had a very rare subclone positive for both loxP sites (conditional knock-out allele), possibly resulting from subsequent re-targeting of the previously targeted allele. These results confirm that using a donor with low homology to the endogenous exon is most efficient at generating conditional knock-out alleles. Table 6 provides an overall summary of the data from the microinjection and ES cell experiments.

TABLE 6
Overview of Lrp5 alleles derived from CKO donor plasmid.

| | Allele 1 | Allele 2 | Allele 3 | Allele 4 | Allele 5 | Random |
|---|---|---|---|---|---|---|
| Donor 1 | | | | | | |
| Mouse #95 | 2 bp del. | plasmid int. | — | — | — | no |
| Mouse #140 | CKO | wt | — | — | — | yes |
| Mouse #155 | CKO | wt | 5′loxP | — | — | no |
| Mouse #243 | 3 bp del. | CKO | 1 bp del | — | — | no |
| ES cell C8 | CKO | 9 bp del. | wt | — | — | yes |
| Donor 2 | | | | | | |
| Mouse #250 | 27 bp del. | 1 bp del. | CKO | wt | 3′ loxP w/ | no |
| | | | | | 27 bp del. | |
| Mouse #274 | 3′ loxP | 1 bp del. | — | — | — | no |
| Mouse #280 | wt | donor only | plasmid int. | — | — | no |
| ES cell F5 | wt | 5′ loxP | donor only | — | — | no |
| ES cell H10 | wt | 3′ loxP | 14 bp deletion | — | — | yes |
| Donor 3 | | | | | | |
| ES cell E3 | 95 bp del. | 5′ loxP | wt | plasmid int. | — | no |
| ES cell E4 | wt | 5′loxP | 4 bp deletion | CKO | — | no |

### Example 5

### Normal Gene Function of Conditional Knock-Out Allele

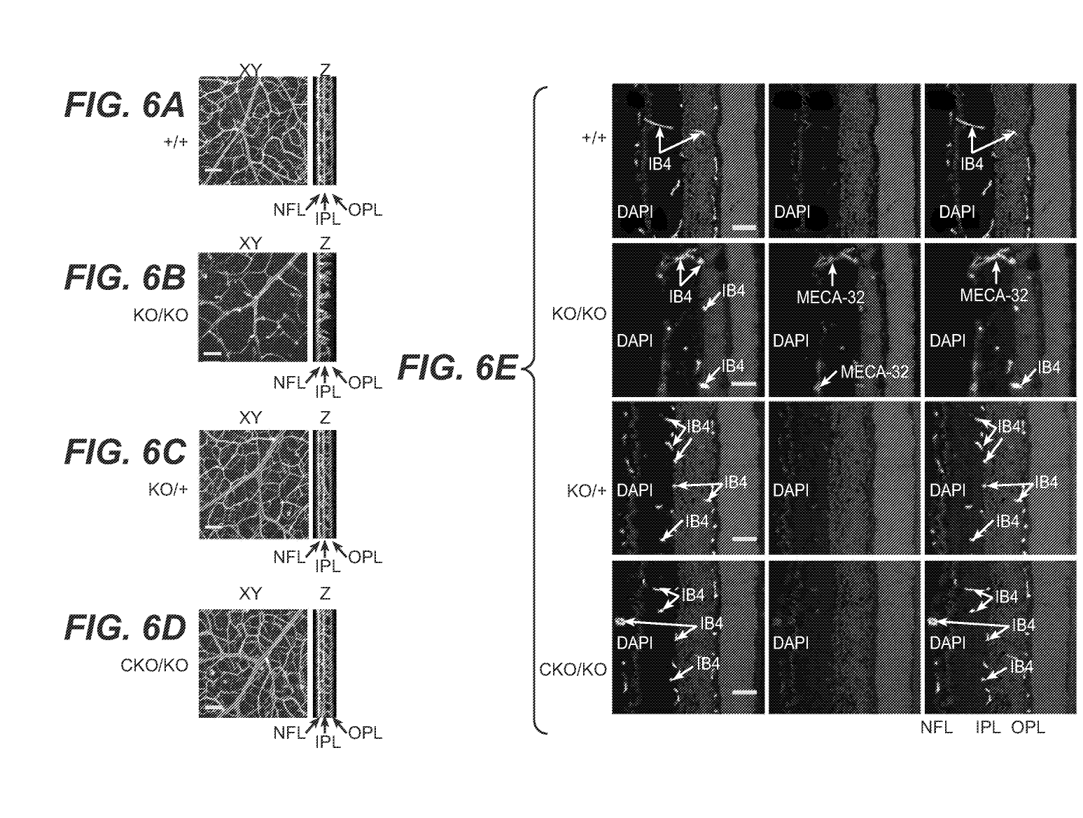

To determine if the silent mutations in the conditional knock-out allele obtained from donor 1 (FIG. 4A; FIG. 5) affected normal function of the Lrp5 gene, mice carrying one knock-out allele (#140) and one conditional knock-out allele (#155) were bred with Lrp5 knock-out homozygous mice generated using the ZFN pair of Example 3. Age matched postnatal day 16 (P16) control mice (FIG. 6A, +/+) were derived from an Lrp5 heterozygous cross. The other mice used for the experiments were derived from a cross between an Lrp5 KO/KO female and an Lrp5 CKO/+ male. The Lrp5 KO/KO female (FIG. 6B) is the adult mother of KO/+ (FIG. 6C, P16) and CKO/KO (FIG. 6D, P16). FIGS. 6A-D show representative confocal projections of retinal whole mounts stained with isolectin B4 (IB4) (scale bars: 50 μm). For each projection shown in FIGS. 6A-D, the left image depicts the maximum XY projection and the right image depicts the Z projection displaying vasculatures in the nerve fiber layer (NFL), the inner plexiform layer (IPL), and the outer plexiform layer (OPL) (labels on the bottom right panel of FIG. 6D). The Lrp5 null animal showed a reduced vascular complexity in the XY projection and the absence of deep vascular layers (FIG. 6B). Mice carrying the conditional knock-out allele on the null background display a normal vascular phenotype (FIG. 6D), suggesting that the conditional knock-out allele is functional. FIG. 6E shows retinal cross sections of the opposite eyes to those depicted in FIGS. 6A-D stained with IB4, MECA32, and DAPI. Homozygous knock-out mice ectopically expressed the fenestrated endothelial cell marker MECA32, whereas the CKO/KO, KO/+, and +/+ mice are MECA32 negative. In summary, homozygous knock-out animals display the retinal phenotypes described above (FIG. 6), whereas the retinal phenotypes of mice carrying one knock-out allele and one conditional knock-out allele were indistinguishable from those of wild type mice or mice having either one knock-out allele and one wild type allele (FIG. 6), indicating that the conditional knock-out allele is a functional allele. Together these results demonstrate that a recombinase-recognition site-flanked donor sequence having neutral mutations can be used together with a sequence-specific nuclease to generate fully functional conditional knock out alleles in vitro and in vivo.

FIG. 7 illustrates possible mechanism that gave rise to the Lrp5 alleles observed in these studies. The overall homology between Lrp5 genomic sequence and donor 1 is reduced by multiple silent mutations (FIG. 7A, asterisks). After resection of chromosome ends, strand invasion takes place in the large regions of 100% homology outside of the loxP sites, leading to a conditional knock-out allele having both loxP sites. Due to the limited homology in the region between the loxP sites, cross-over events inside the loxP sites is rare. Donor 2 contains larger regions of 100% homology between the loxP sites, allowing for strand invasion to take place inside of the loxP sites, resulting in alleles having a 3′ loxP site only (FIG. 7B), a 5′ loxP site only (FIG. 7C), or no loxP site (FIG. 7D). Primer combinations P9+P10 and P11+P12 both gave rise to PCR products for events according to FIG. 7A. Use of primer pair P9+P10 resulted in a product for events depicted in FIG. 7C but not for events depicted in FIG. 7B or

D. Similarly, primer pairs P11+P12 gave rise to a product for events depicted in FIG. 7B but not for events depicted in FIG. 7C or D. Primer combinations P5+P6 and P7+P8 resulted in PCR products regardless of loxP status.

TABLE 7
Conditions for PCR reactions used in the Examples described above.

| Primer | | Cycling Reaction Conditions | | | | Product |
|---|---|---|---|---|---|---|
| Pair | Reagents Used | 95° C. | Annealing | 72° C. | Cycles | Size (bp) |
| P1/P2 | REDExtract-N-Amp PCR ReadyMix | 45 sec | a) 62 C. (−0.3 C./cycle) - 30 sec | 1 min | a) 25 | 1059 |
| | (Sigma, Cat# R4775) | | b) 57 C. - 30 sec | | b) 12 | |
| P3/P4 | REDExtract-N-Amp PCR ReadyMix | 45 sec | a) 56 C. (−0.3 C./cycle) - 30 sec | 30 sec | a) 10 | 370 |
| | (Sigma, Cat# R4775) | | b) 53 C. - 30 sec | | b) 30 | |
| P5/P6 | Advantage GC 2 PCR kit | 45 sec | 56 C. - 45 sec | 1 min 30 sec | 40 | 1394 |
| | (Clontech, Cat# 639119) | | | | | |
| P7/P8 | REDExtract-N-Amp PCR ReadyMix | 45 sec | 63 C. - 45 sec | 1 min 30 sec | 40 | 1410 |
| | (Sigma, Cat# R4775) | | | | | |
| P9/P10 | Advantage GC 2 PCR kit | 45 sec | 56.5 C. - 45 sec | 1 min 30 sec | 40 | 1195 |
| | (Clontech, Cat# 639119) | | | | | |
| P11/P12 | REDExtract-N-Amp PCR ReadyMix | 45 sec | 62 C. - 45 sec | 1 min 30 sec | 40 | 1105 |
| | (Sigma, Cat# R4775) | | | | | |
| P13/P6 | Advantage GC 2 PCR kit | 45 sec | 56 C. - 45 sec | 1 min 30 sec | 40 | 1482 |
| | (Clontech, Cat# 639119) | | | | | |
| P7/P14 | REDExtract-N-Amp PCR ReadyMix | 45 sec | 62 C. - 45 sec | 1 min 30 sec | 40 | 1462 |
| | (Sigma, Cat# R4775) | | | | | |
| P5/P14 | LA Taq | 45 sec | 55 C. - 45 sec | 10 min | 47 | 2836 |
| | (TaKaRa, Cat# RR002M) | | | | | |
| P13/P8 | LA Taq | 45 sec | 55 C. - 45 sec | 10 min | 47 | 2871 |
| | (TaKaRa, Cat# RR002M) | | | | | |
| P5/P8 | Advantage GC 2 PCR kit | 45 sec | 57 C. - 45 sec | 3 min 30 sec | 40 | wt - 2729 |
| | (Clontech, Cat# 639119) | | | | | CKO - 2797 |
| P13/P15 | Advantage GC 2 PCR kit | 45 sec | 56 C. - 45 sec | 1 min 30 sec | 40 | 1273 |
| | (Clontech, Cat# 639119) | | | | | |
| P16/P14 | REDExtract-N-Amp PCR ReadyMix | 45 sec | 63 C. - 45 sec | 1 min 30 sec | 40 | 1211 |
| | (Sigma, Cat# R4775) | | | | | |
| P17/P18 | Type-it Fast SNP Probe PCR mix | 15 sec | 60 C. - 60 sec | 20 sec | 40 | fluorescence |
| P19 | (Qiagen, Cat# 206042) | | | | | |

TABLE 8
Conservative substitutions.

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

### Example 6

### Cas9/CRISPR-Mediated Mutagenesis of Lrp5 Exon 2 Using Different Guide RNAs

To confirm that other sequence-specific endonucleases can be used with the methods and compositions described herein, modified alleles of Lrp5 were produced using the Cas9/CRISPR system. Hepa1-6 murine hepatoma cells were cultured in RPMI supplemented with 10% FBS, L-glutamine, and antibiotics. After trypsinization and pelleting, 10⁶ cells were electroporated with 2 μg per plasmid containing hCas9-encoding cDNA or 15 μg of mRNA encoding Cas9 (FIG. 14, SEQ ID NO: 43) using AMAXA Nucleofector Kit V with AMAXA Nucleofector program T-028 (Lonza) according to the manufacturer's instruction and plated into a 6 well plate. Nucleofection efficiencies reached 80-95% as assessed by GFP expression (PMAXGFP). Fresh media was exchanged at 24 hrs post-nucleofection and purified genomic DNA was harvested at 72 hr post-nucleofection using DNeasy Blood and Tissue kit (Qiagen). HCas9 mRNA was transcribed in vitro with MMESSAGE MMachine T7 Ultra kit (Life Technologies) following the manufacturer's protocol, including a polyA tailing reaction. mRNA was purified and concentrated using standard phenol:chloroform extraction and precipitation of RNA.

Three unique guide RNAs (gRNAs) targeting mouse Lrp5 exon 2 were generated (FIG. 14A; Lrp5 gRNA T2, Lrp5 gRNA T5 and Lrp5 gRNA T7; SEQ ID NOS: 36-38).

NIH/3T3 cells or Hepa1-6 cells were co-transfected with either DNA encoding zinc finger pairs (pZFN1+pZFN2) or with Cas9 (+pRK5-hCas9) together with a guide RNA targeting Lrp5 exon 2 (p_gRNA T2, p_gRNA T5 or p_gRNA T7) or a control plasmid (PMAXGFP). gRNA T7 sequence overlaps with the 3′ end of the right ZFN protein binding site sequence.

To detect mutations in the Lrp5 locus following co-transfection, indicative of Cas9-mediated cleavage and subsequent repair, SURVEYOR Assays (Transgenomic) were performed essentially according to the manufacturer's instruction. In this assay, PCR products are hybridized. In the event of mutations, the hybridization complex contains a mismatch which is cleaved by the SURVEYOR nuclease. In this example, a ˜2.7 kb PCR product specific for the Lrp5 exon2 genomic locus was amplified using primers P9 and P12 (SEQ ID NOS: 9 and 12) using the following parameters and LA Taq (Takara): 95° C. for 3 min, 35 cycles of 95° C. for 45 sec; 57° C. for 45 sec; 70° C. for 2 min 30 sec, followed by 72° C. for 7 min. One-third of the PCR product was used in the SURVEYOR Assay. Resulting digested products were resolved by electrophoresis on a 1.5% agarose gel. Nuclease cutting was identified by the presence of shorter fragments, which indicated the presence of mutant alleles that annealed with wildtype.

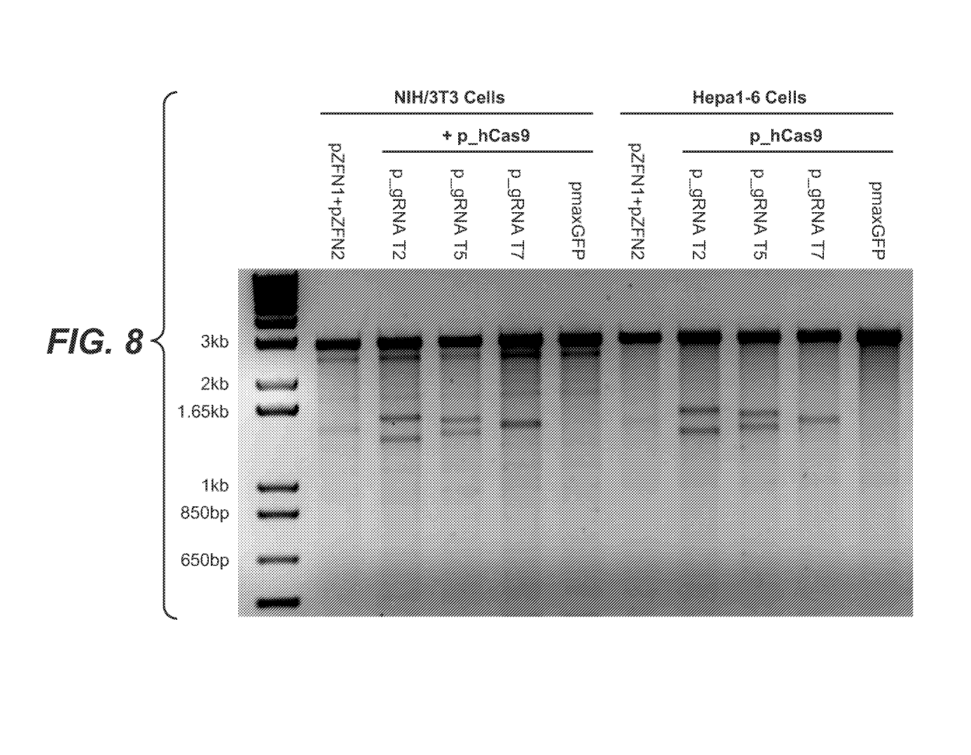

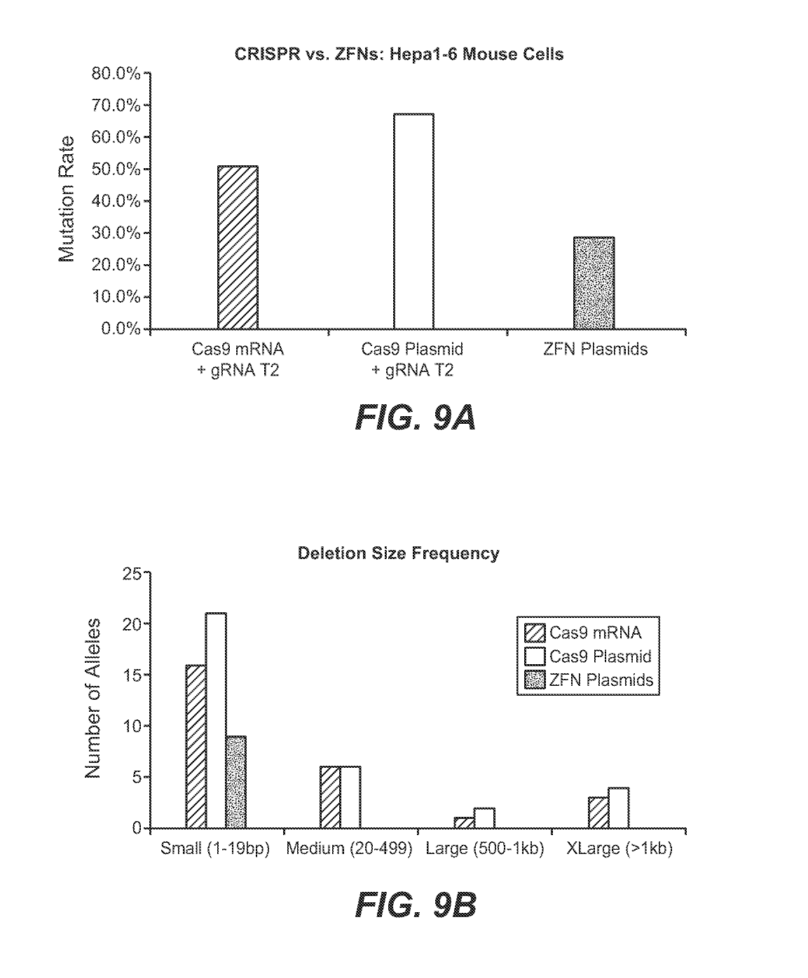

All three guide RNAs (gRNAs) targeting mouse Lrp5 exon 2 efficiently mediated Cas9-induced mutations (FIG. 8). The activity of each gRNA/Cas9 pairing appears to be folds greater than ZFN mediated mutagenesis in these experiments. Mutation rates were calculated from sequencing TOPO cloned alleles from a 2.7 kb PCR product of the Lrp5 exon 2 genomic locus. Alignments of individual sequences to wildtype determined exact deletion (quantified above) or insertion sizes (data not shown). A 2.7 kb genomic region was amplified by PCR with primers P9 and P12 as described above. The PCR products were cloned directly using TOPO-TA cloning (Invitrogen) to capture all possible deletion sizes. After transformation and plating for single colonies, clones were selected, plasmid DNA isolated, and sequenced according to the Sanger method using primers P20 and P21. FIG. 9A-B illustrate a summary of gRNA/Cas9 mutation rates (FIG. 9A) and deletion sizes (FIG. 9B) in Hepa1-6 murine hepatoma cells.

### Example 7

### Cas9/CRISPR Mediated Gene Targeting Using a Codon-Optimized Conditional Knock-Out Donor Vector

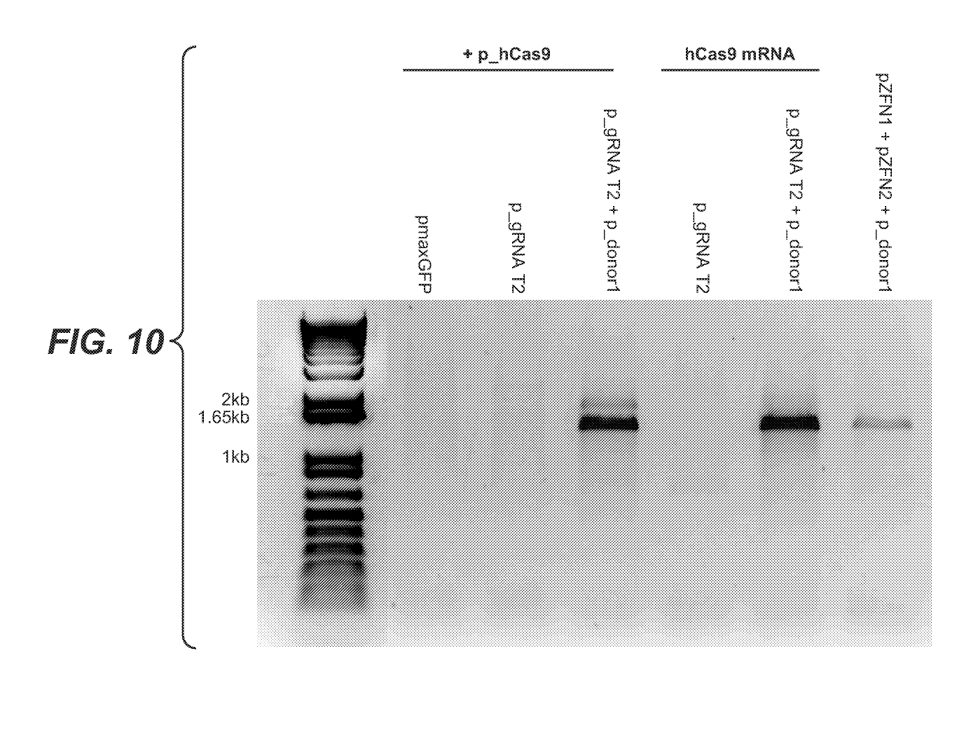

Hepa1-6 cells were co-transfected with Cas9 plasmid or mRNA, a gRNA and the Lrp5 CKO donor 1 comprising the codon-optimized exon sequence. For comparison, some cells were co-transfected with Lrp5 ZFN plasmids and the donor plasmid (FIG. 10). After 72 hours, genomic DNA from the transfected cells was analyzed by PCR with a primer specific to the codon-optimized Lrp5 donor exon (P7; SEQ ID NO: 7), and a primer specific to a region outside of the 3′ homology arm (P12; SEQ ID NO: 12). Primers P7 and P12 were used for the PCR reaction with REDExtract-N-Amp PCR ReadyMix (Sigma) with the following conditions: 95° C. for 3 min, 38 cycles of 95° C. for 45 sec; 63° C. for 45 sec; 72° C. for 1 min 30 sec, followed by 72° C. for 7 min. PCR products were resolved by electrophoresis on a 1% agarose gel. As described above, the Lrp5 exon 2 donor1 vector contains a codon optimized exon (COexon2) harboring many neutral mutations, excluding from mutation the first 13 bp and the last 11 bp, as well as exogenous flanking loxP sites. The PCR above uses a forward primer specific for COexon2 sequence and a reverse primer outside of the homology arm in the genomic locus, therefore producing a PCR product only if the donor exon sequence was incorporated in the correct Lrp5 locus. The use of gRNA/Cas9 resulted in donor sequence integration at the Lrp5 locus with great efficiency, exceeding that observed when using the ZFN system and the same donor vector strategy (FIG. 10).

### Example 8

### Cas9/CRISPR Mediated Targeted Introduction of loxP Sites

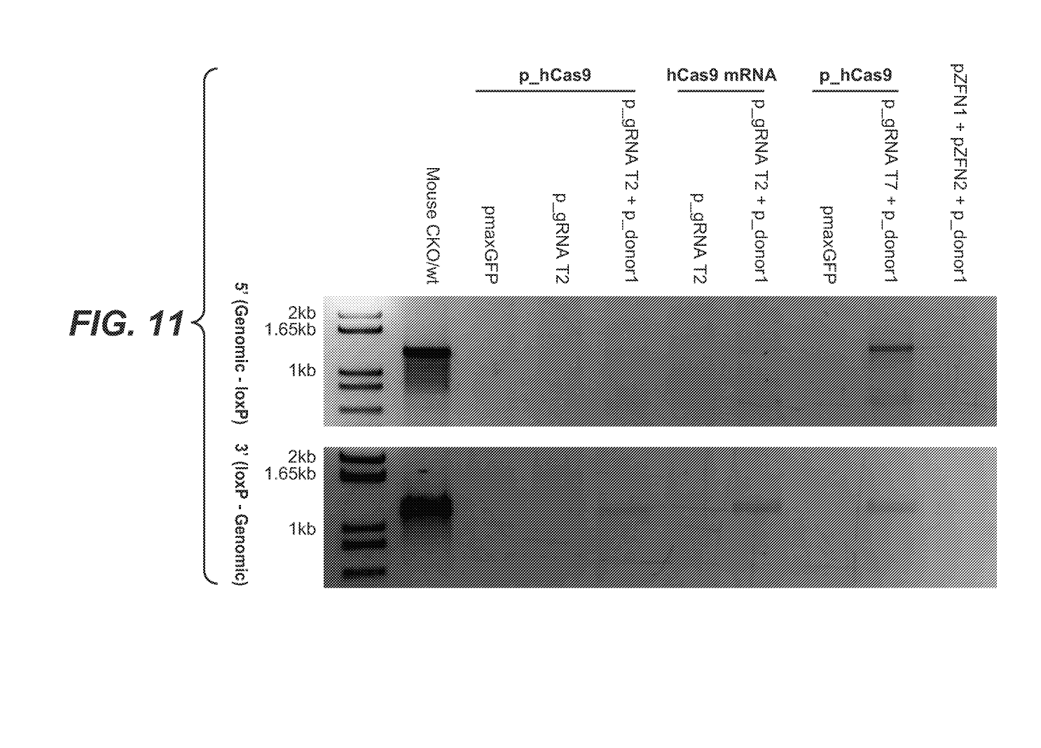

To determine if the donor design strategy and the Cas9/CRISPR system can be used to introduce loxP sites at a genomic locus, genomic DNA from cells transfected as described in Example 7 was analyzed by PCR analysis using one primer located outside the homology arms, and one primer anchored at either the 5′ or 3′ loxP sites from the donor. For the 5′ genomic to 5′ loxP reaction, primers P9 and P10 (SEQ ID NOS: 9 and 10) were used with the standard Expand High Fidelity PCR System (Roche) protocol except for an addition of DMSO to a final concentration of 2%. PCR parameters were as follows: 95° C. for 3 min, 45 cycles of 95° C. for 45 sec; 63° C. for 45 sec; 72° C. for 1 min 30 sec, followed by 72° C. for 7 min. For the 3′ loxP to 3′ genomic reaction, primers P11 and P12 were used following the standard REDExtract-N-Amp PCR ReadyMix (Sigma) protocol. PCR parameters were as follows: 95° C. for 3 min, 40 cycles of 95° C. for 45 sec; 62.5° C. for 45 sec; 72° C. for 1 min 30 sec, followed by 72° C. for 7 min. PCR products were resolved by electrophoresis on 1% agarose gels. PCR products were obtained for the 3′ loxP site from samples isolated from cells that were transfected with either of the two different Lrp5 gRNAs and the CKO donor (FIG. 11; p_gRNA T2). Similarly, PCR products were obtained from samples isolated from cells that were transfected with gRNA T7 for the 5′ loxP site (FIG. 11). Thus, FIG. 11 shows that in Hepa1-6 cells, Lrp5 gRNA T2/Cas9 and Lrp5 gRNA T7/Cas9 mediated double-strand breaks resulted in introduction of loxP sites at the Lrp5 locus using the codon optimized exon donor vector strategy. Only cells electroporated with Cas9, gRNA, and donor exhibit evidence of 5′ (FIG. 11, top) and 3′ (FIG. 11, bottom) loxP sites in the Lrp5 genomic locus. gRNA T7 resulted in more prominent 5′loxP presence whereas integrated loxP sites were not detectable with ZFNs. The absence of detectable loxP sites in the ZFN samples and low levels in the gRNA samples in these experiments using Hepa1-6 cells might be explained by both low homologous recombination rates in cell lines and the fact that the full cell pool transfected, not clonal subsets, were analyzed. A single mouse genomic DNA sample with an Lrp5 CKO/wt genotype was used as a positive control. These results show that the CKO design strategy can be used in somatic cells and that it effectively reduces the frequency of undesirable cross-over events between the double strand break and the location of both the 5′ and 3′ loxP sites. In summary, targeting of specific genomic loci by introducing RNA-guided nuclease-mediated DNA breaks that are subsequently repaired using an engineered codon-optimized CKO donor sequence can be used to insert loxP sites and thereby produce conditional knock-out alleles.

### Example 9

### Targeting of Usp10, Nnmt, and Notch3 Genomic Loci

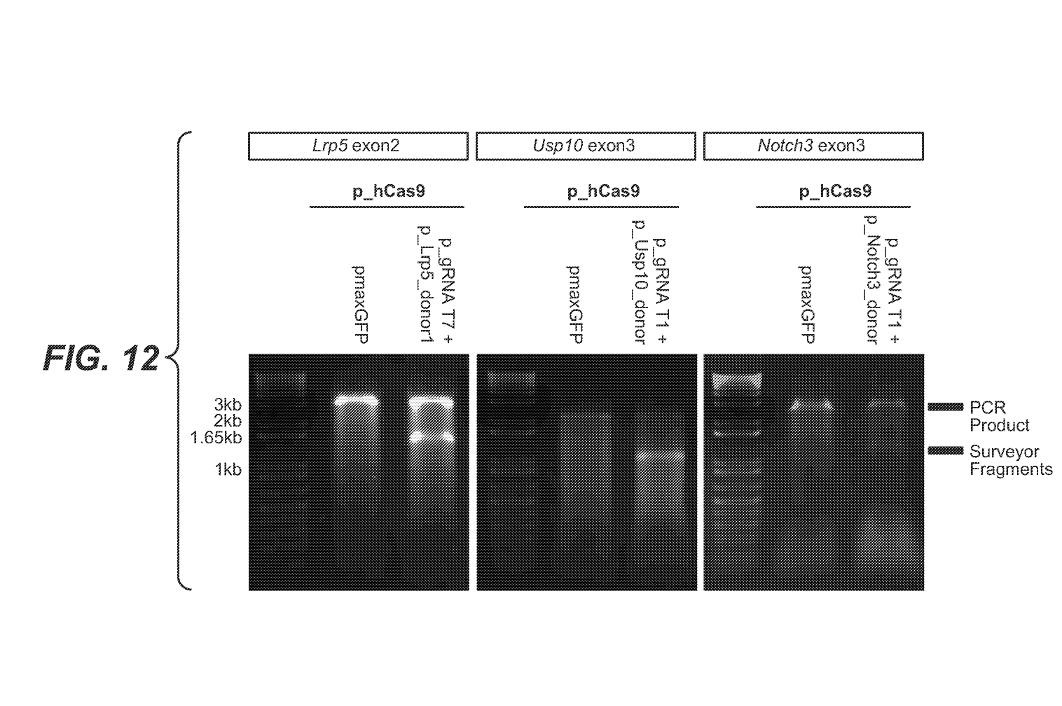

To confirm that other genes can be targeted with the inventive methods, donor and gRNAs for the Usp10, Nnmt, and Notch3 genomic loci were generated. These Cas9/gRNAs and donors were introduced into Hepa1-6 cells as described in Example 6 and as depicted in FIG. 12 to introduce DNA double-strand breaks at the respective loci and subsequent repair using the codon-optimized donor as a template. A SURVEYOR Assays were performed essentially as described above. PCR products 2.2-2.7 kb in size, specific for Lrp5, Usp10, and Notch3 genomic loci were amplified using primers P9, P12, P22, P23, P24, P25 (SEQ ID NOS: 9, 12, 22, 23, 24 and 25, respectively) and the following parameters with LA Taq (Takara): 95° C. for 3 min, 35 cycles of 95° C. for 45 sec; Ta for 45 sec (Lrp5=57 C, Usp10 & Notch3=63); 70° C. for 2 min 30 sec, followed by 72° C. for 7 min. One-seventh, ⅓, and all of the PCR products were used, respectively, in the SURVEYOR Assay as following the manufacturer's instruction (Transgenomic). Resulting digested products representing nuclease cutting where strands of wildtype and mutant alleles have annealed, were resolved by electrophoresis on a 1.5% agarose gel

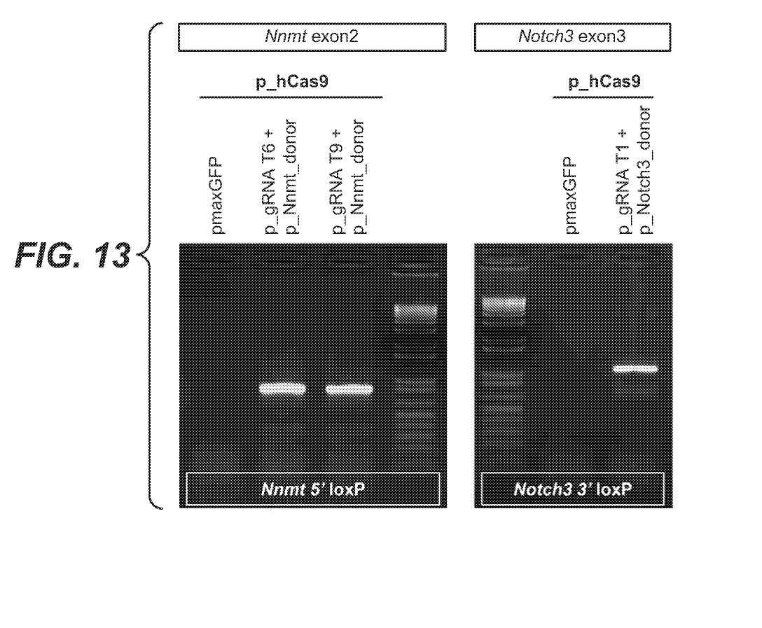

FIG. 12 and FIG. 13 show that as observed with the Lrp5 locus, Usp10, Nnmt, and Notch3 genomic loci were efficiently targeted by specific gRNA/Cas9 complexes (FIG. 12) and that loxP sites were integrated (FIG. 13).

### Example 10

### Generation of Conditional Knock-Out and Knock Out Alleles of Lrp5 Using RNA-Guided Sequence-Specific Endonucleases and Codon-Optimized Donor

The Lrp5 locus can be targeted with the Lrp5-specific gRNAs described herein to introduce a floxed codon-optimized exon thereby creating conditional knock-out alleles. Subsequent expression of the Cre recombinase protein in cells harboring the conditional knock out allele can excise the floxed exon resulting in a knock-out allele.

TABLE 9
Primer nucleotide sequences.

| Primer | | |
|---|---|---|
| Number | Primer Sequence (5′−>3′) | Purpose |
| 20 | AGG AAA GCT AGC TTT CCA GGA GTA TG | Sequencing Lrp5 genomic PCR |
| | (SEQ ID NO: 20) | |
| 21 | GGA AGT CAA ATC CTC CTG GTT ACG A | Sequencing Lrp5 genomic PCR |
| | (SEQ ID NO: 21) | |
| 22 | GGC GTC CAG ATT ATG CAC AC | Amplify Usp10 locus |
| | (SEQ ID NO: 22) | |
| 23 | GAT AAT CAT GGA ATC TAA TC | Amplify Usp10 locus |
| | (SEQ ID NO: 23) | |
| 24 | TCT TTG CCT GAC CTG GCT ATG AG | Amplify Notch3 locus |
| | (SEQ ID NO: 24) | |
| 25 | CAA TCT TTC TAA CGC TCA ACT CAG AGT C | Amplify Notch3 locus/Detect 3′loxp |
| | (SEQ ID NO: 25) | |
| 26 | CAT TGG GCT GGT ACA CGG A | Detect Nnmt 5′loxp |
| | (SEQ ID NO: 26) | |
| 27 | GAG CTG AAG TTA TAG ATA CT TCG TAT AGC | Detect Nnmt 5′loxP |
| | (SEQ ID NO: 27) | |
| 28 | GGG AAC CCT ATA ACT TCG TAT AAT G | Detect Notch3 3′loxP |
| | (SEQ ID NO: 28) | |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of generating a conditional knock-out allele in a cell comprising a target gene, the method comprising the steps of:
a. introducing into the cell a donor construct, wherein the donor construct comprises a 5′ homology region, a 5′ recombinase recognition site, a donor sequence, a 3′ recombinase recognition site, and a 3′ homology region, wherein the donor sequence comprises a target sequence having at least one neutral mutation; and
b. introducing into the cell a sequence-specific nuclease that cleaves a sequence within the target gene, thereby producing a conditional knock-out allele in the cell.

2. The method of claim 1, wherein the sequence-specific nuclease is a zinc finger nuclease (ZFN).

3. The method of claim 1, wherein the sequence-specific nuclease is a transcription activator-like effector nuclease (TALEN).

4. The method of claim 1, wherein the sequence-specific nuclease is a ZFN dimer that cleaves the target gene only once.

5. The method of claim 1, wherein the sequence-specific nuclease is an RNA-guided nuclease.

6. The method of claim 5, wherein the RNA-guided nuclease is Cas9.

7. The method of claim 1, wherein the sequence-specific nuclease is introduced as a protein, mRNA, or cDNA.

8. The method of claim 1, wherein the recombinase recognition site is a loxP site, an frt site, or a rox site.

9. The method of claim 1, wherein the donor sequence comprises seven silent mutations.

10. The method of claim 1, wherein sequence homology between the donor sequence and the target sequence is 98% or less.

11. The method of claim 10, wherein sequence homology between the donor sequence and the target sequence is 78%.

12. The method of claim 1, wherein the donor construct comprises the sequence of SEQ ID NO: 30, 31, 44, 45, or 46.

13. The method of claim 1, wherein the 5′ homology region comprises at least 1.1 kb and wherein the 3′ homology region comprises at least 1 kb.

14. The method of claim 1, wherein the target gene is selected from the group consisting of Lrp5, Usp10, Nnmt, and Notch3.

15. The method of claim 1, wherein the cell was isolated from a mammal.

16. The method of claim 15, wherein the mammal is selected from the group consisting of mouse, rat, rabbit, hamster, guinea pig, cat, dog, sheep, horse, cow, monkey, and human.

17. The method of claim 1, wherein the cell is a zygote or a pluripotent stem cell.

18. A method of generating a conditional knock-out animal, the method comprising the steps of:
a. introducing a donor construct into a cell comprising a target gene, wherein the donor construct comprises a 5′ homology region, a 5′ recombinase recognition site, a donor sequence, a 3′ recombinase recognition site, and a 3′ homology region, wherein the donor sequence comprises a target sequence having at least one neutral mutation;
b. introducing a sequence-specific nuclease into the cell, wherein the nuclease cleaves the target gene; and
c. introducing the cell into a carrier animal to produce the conditional knock-out animal from the cell.

19. The method of claim 18, wherein the cell is a zygote or a pluripotent stem cell.

20. A method of generating a knock-out animal, the method comprising the steps of:
a. introducing a donor construct into a cell comprising a target gene, wherein the donor construct comprises a 5′ homology region, a 5′ recombinase recognition site, a donor sequence, a 3′ recombinase recognition site, and a 3′ homology region, wherein the donor sequence comprises a target sequence having at least one neutral mutation;
b. introducing a sequence-specific nuclease into the cell, wherein the nuclease cleaves the target gene;
c. introducing the cell into a carrier animal to produce a transgenic animal from the transfected cell; and
d. breeding the conditional knock-out animal with a transgenic animal having a transgene encoding a recombinase protein that catalyzes recombination at the 5′ and 3′ recombinase recognition site.

21. The method of claim 20, wherein the cell is a zygote or a pluripotent stem cell.

22. The method of claim 20, wherein the recombinase recognition site is a loxP site and wherein the recombinase is Cre recombinase.

23. The method of claim 20, wherein the recombinase recognition site is an frt site and wherein the recombinase is FLP recombinase.

24. The method of claim 20, wherein the recombinase recognition site is a rox site and wherein the recombinase is Dre recombinase.

25. The method of claim 20, wherein the transgene encoding the recombinase is under the control of a tissue-specific promoter or an inducible promoter.

26. A composition for generating a conditional knock-out allele of a target gene comprising:
a. a donor construct comprising a 5′ homology region, a 5′ recombinase recognition site, a donor sequence, a 3′ recombinase recognition site, and a 3′ homology region, wherein the donor sequence comprises a target sequence having at least one neutral mutation compared to the sequence of the target gene; and
b. a sequence-specific nuclease that recognizes the target gene.

27. The composition of claim 26, wherein the sequence-specific nuclease is selected from the group consisting of ZFN, TALEN, and RNA-guided nuclease.

28. A donor construct comprising the sequence of SEQ ID NO: 30, 31, 44, 45, or 46.

29. A cell comprising the donor construct of claim 28.

30. A non-human conditional knock-out animal prepared according to the method of claim 18.


SEQUENCE LISTING (46 sequences)

SEQ ID NO: 1 - DNA, 23 bp, Artificial Sequence
  Description of Artificial Sequence Synthetic primer
catgtgcctttgaagagcacacc

SEQ ID NO: 2 - DNA, 24 bp, Artificial Sequence
  Description of Artificial Sequence Synthetic primer
actccacggtcctgggattataga

SEQ ID NO: 3 - DNA, 20 bp, Artificial Sequence
  Description of Artificial Sequence Synthetic primer
ggcctatcactaagggagcc

SEQ ID NO: 4 - DNA, 20 bp, Artificial Sequence
  Description of Artificial Sequence Synthetic primer
gcccgagatgacaatgttct

SEQ ID NO: 5 - DNA, 26 bp, Artificial Sequence
  Description of Artificial Sequence Synthetic primer
cgagcttttcttagtgatcttttaag

SEQ ID NO: 6 - DNA, 21 bp, Artificial Sequence
  Description of Artificial Sequence Synthetic primer
ctcacgtcggtccaataaacg

SEQ ID NO: 7 - DNA, 21 bp, Artificial Sequence
  Description of Artificial Sequence Synthetic primer
cgtttattggaccgacgtgag

SEQ ID NO: 8 - DNA, 22 bp, Artificial Sequence
  Description of Artificial Sequence Synthetic primer
cctagactgcagtgaaggacat

SEQ ID NO: 9 - DNA, 32 bp, Artificial Sequence
  Description of Artificial Sequence Synthetic primer
gctcacgagcttttcttagtgatcttttaagg

SEQ ID NO: 10 - DNA, 30 bp, Artificial Sequence
  Description of Artificial Sequence Synthetic primer
gagaatcatgcacggataacttcgtatagc

SEQ ID NO: 11 - DNA, 36 bp, Artificial Sequence
  Description of Artificial Sequence Synthetic primer
caggatttcttctgtagagtataacttcgtataatg

SEQ ID NO: 12 - DNA, 26 bp, Artificial Sequence
  Description of Artificial Sequence Synthetic primer
cctagactgcagtgaaggacattcac

SEQ ID NO: 13 - DNA, 29 bp, Artificial Sequence
  Description of Artificial Sequence Synthetic primer
ggataacaatttcacacaggaaacagcta

SEQ ID NO: 14 - DNA, 24 bp, Artificial Sequence
  Description of Artificial Sequence Synthetic primer
gtaaaacgacggccagtgaattgg

SEQ ID NO: 15 - DNA, 21 bp, Artificial Sequence
  Description of Artificial Sequence Synthetic primer
cagggaaagagaatcatgcac

SEQ ID NO: 16 - DNA, 21 bp, Artificial Sequence
  Description of Artificial Sequence Synthetic primer
ctgcacatgggtaaacctctg

SEQ ID NO: 17 - DNA, 18 bp, Artificial Sequence
  Description of Artificial Sequence Synthetic primer
cacctgaactactgaaag

SEQ ID NO: 18 - DNA, 18 bp, Artificial Sequence
  Description of Artificial Sequence Synthetic primer
cagggaaagagaatcatg

SEQ ID NO: 19 - DNA, 17 bp, Artificial Sequence
  Description of Artificial Sequence Synthetic primer
tatagcatacattatac

SEQ ID NO: 20 - DNA, 26 bp, Artificial Sequence
  Description of Artificial Sequence Synthetic primer
aggaaagctagctttccaggagtatg

SEQ ID NO: 21 - DNA, 25 bp, Artificial Sequence
  Description of Artificial Sequence Synthetic primer
ggaagtcaaatcctcctggttacga

SEQ ID NO: 22 - DNA, 20 bp, Artificial Sequence
  Description of Artificial Sequence Synthetic primer
ggcgtccagattatgcacac

SEQ ID NO: 23 - DNA, 20 bp, Artificial Sequence
  Description of Artificial Sequence Synthetic primer
gataatcatggaatctaatc

SEQ ID NO: 24 - DNA, 23 bp, Artificial Sequence
  Description of Artificial Sequence Synthetic primer
tctttgcctgacctggctatgag

SEQ ID NO: 25 - DNA, 28 bp, Artificial Sequence
  Description of Artificial Sequence Synthetic primer
caatctttctaacgctcaactcagagtc

SEQ ID NO: 26 - DNA, 19 bp, Artificial Sequence
  Description of Artificial Sequence Synthetic primer
cattgggctggtacacgga

SEQ ID NO: 27 - DNA, 30 bp, Artificial Sequence
  Description of Artificial Sequence Synthetic primer
gagctgaagttatagataacttcgtatagc

SEQ ID NO: 28 - DNA, 25 bp, Artificial Sequence
  Description of Artificial Sequence Synthetic primer
gggaaccctataacttcgtataatg

SEQ ID NO: 29 - DNA, 39 bp, Artificial Sequence
  Description of Artificial Sequence Synthetic oligonucleotide
gacttccagttctccaagggtgctgtgtactggacagat

SEQ ID NO: 30 - DNA, 5948 bp, Artificial Sequence
  Description of Artificial Sequence Synthetic polynucleotide
aatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctccttcctcttccagcccttcctctttcactgactgactgactggaagacacacctgcggccgcgcaagctgggccatggggcttaacccctaacatcactcttggtgtgacttggcacaagtgtatgcttctttatgaaggctgcagctctgggcctggagaatgtataccaggacccctagtctaccaggggctgtgcatttgagaggcatgggtcacctggtaggggaggaacccgtaggaccagcctcctgactgaggctggcactgtgtgtttctaccctgggaactttgcatgggttttaatgaggcctcccctaaccactgtatgtagtgtgcagccttctcctgttctgttcccactcctgtcctgtccctgactctggcccattcacagcattaacctgacagatgtcactacctgcttttggtctttgcccggtgtctgtttctccactggaacgggaaccttggaggcccctggtacaagcagcccctgcttgccagcctatgaaggcatcttggttttgccactgtggctctttttctttcactcccagaactttctttttctcctcctgagctttcctttgcagcttctgcccttgaaaaggagaggctggtggagccttggggtgatacagtgtctgtgttgtctaggattccccaggcgggggaggagaggagaagcagcctgccccaggtgagggtggggagccacccacattctggcctgcctcactgtgggtccctggggccactgccaggcctcttagaaggaaagctagctttccaggagtatgctgtgggttccccttaaaacttagggattaagtgtgtggtttctttacttttcagagctgggagcactgccccaccatctcccagtgagggttggagaaaaaaaaagtcctaggttacaaatccacccccagcctccctgtccctgcccctgccccctcccctcccatgtgcctttgaagagcacacctgaactactgaaagcaagttgaggtgaggaagtggtctttccagtttggggagcagtagagccagagcttggggatttggggagccttcctgcctctctatactggcctatcactaagggagcctttgggcaactggaaggaaggagtcttgggagcaggggcccggggtagataacttcgtataatgtatgctatacgaagttatccgtgcatgattctctttccctgggagctggttcctgtcctgcttagccatcctgaccctgtgctctgcccacagcctcaccgctcctcctctttgctaaccgccgagatgttcgactcgtagacgctggcggcgtgaagctggagagtacaatcgttgctagtggactggaggatgctgccgctgtcgacttccagttctcaaagggagccgtttattggaccgacgtgagcgaagaggcaataaaacagacataccttaaccagactggagctgccgcccagaatatcgtaatctctggcttggtgagccctgatggactcgcttgtgattgggtcggaaaaaagctttattggactgatagtgaaacgaatcggatagaagtagcgaaccttaacggtacatcaagaaaggtcctgttttggcaggatcttgaccagcctagagcaatcgccctcgaccctgcacatgggtaaacctctgtttttctcctgctcctatggggaggcgcggggaacaggatttcttctgtagagtataacttcgtataatgtatgctatacgaagttatacctgaggcaggctgatgaggtgccttgggaagcagcccttgctgggaatcacctcccttccagatcctgaactgtaaaacatggcatgtttagccagggatggtggttcacacctacaatcctagcattctggaggctgaggcaggagtgttgccacaagttccaggccaacctgagctataaagaccgtcttaaaaatatacaggaactggggagatgcctccattggtgatgtacttacctcgtaaccaggaggatttgacttccatccatgaagaaagttaggtgcggggcgtcacatgtctataatcccaggaccgtggagttagagacacagggatctgtgaggcttgctggctgcctgcagtcaaattgcccagcccctggtcccagtgagagacaaggtgttttaaggtatttatattgccgtcttaaaaactccatgaccaagaacagcttggggatgaatcagtcagtatacttgatcagacaatactcaagtcatgccccacctctgagtaaagtcagggcaggacctcaaggcaggaacccagagtcaagaactaaactgcttagtggtttgctctccattggttgttcagattgctctcttgtagcacccaggaccatcagcccaggcactgaactgcccacagtgacctgtaccctcccccaccaatcatcagccaaggaaatgcttcccaggcttagaggccaatgcaaactgactgtagcttagctgagcaggacgttgtgtctcaatcaaacacacaaacaagggcagatgaggatgatgcttgggtttgaagcttagcatccttctgcactaggacaggtgacccaggtaagttcagggacctcccaccccttctgtgactgttggctgtggtggctctgtgtctggatgtagcacctactctgtgtattctggaagttacctatattggtgcagtcaactcactctgaccctggaaagcgcaggacagccatgaaggccacgcggccgcacgacagtcttcactgactgactgactggaaagtcctctccactgactgtagcctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcatt

SEQ ID NO: 31 - DNA, 5948 bp, Artificial Sequence
  Description of Artificial Sequence Synthetic polynucleotide
tttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctccttcctcttccagcccttcctctttccagtcagtcagtcagtggagactgtcgtgcggccgcgtggccttcatggctgtcctgcgctttccagggtcagagtgagttgactgcaccaatataggtaacttccagaatacacagagtaggtgctacatccagacacagagccaccacagccaacagtcacagaaggggtgggaggtccctgaacttacctgggtcacctgtcctagtgcagaaggatgctaagcttcaaacccaagcatcatcctcatctgcccttgtttgtgtgtttgattgagacacaacgtcctgctcagctaagctacagtcagtttgcattggcctctaagcctgggaagcatttccttggctgatgattggtgggggagggtacaggtcactgtgggcagttcagtgcctgggctgatggtcctgggtgctacaagagagcaatctgaacaaccaatggagagcaaaccactaagcagtttagttcttgactctgggttcctgccttgaggtcctgccctgactttactcagaggtggggcatgacttgagtattgtctgatcaagtatactgactgattcatccccaagctgttcttggtcatggagtttttaagacggcaatataaataccttaaaacaccttgtctctcactgggaccaggggctgggcaatttgactgcaggcagccagcaagcctcacagatccctgtgtctctaactccacggtcctgggattatagacatgtgacgccccgcacctaactttcttcatggatggaagtcaaatcctcctggttacgaggtaagtacatcaccaatggaggcatctccccagttcctgtatatttttaagacggtctttatagctcaggttggcctggaacttgtggcaacactcctgcctcagcctccagaatgctaggattgtaggtgtgaaccaccatccctggctaaacatgccatgttttacagttcaggatctggaagggaggtgattcccagcaagggctgcttcccaaggcacctcatcagcctgcctcaggtataacttcgtatagcatacattatacgaagttatactctacagaagaaatcctgttccccgcgcctccccataggagcaggagaaaaacagaggtttacccatgtgcaggatccagggcaatggcccttggctggtccaggtcctgccagaagagaaccttacgggacgtcccattgaggttggcaacctcaatgcggttggtctcggagtccgtccagtacagcttcttgccaacccagtcacaggccaggccatcaggtgacacgaggcccgagatgacaatgttctgtgcagcagctccagtctggttcaggtaggtctgtttgatggcctcctcgctcacgtcggtccaataaacggctccctttgagaactggaagtctacagcagctgcatcctccaggccactggccacaatggtggactccagcttcactccgccggcatccactagccgcacatcccggcggttggcaaacaacaggagcggtgaggctgtgggcagagcacagggtcaggatggctaagcaggacaggaaccagctcccagggaaagagaatcatgcacggataacttcgtatagcatacattatacgaagttatctaccccgggcccctgctcccaagactccttccttccagttgcccaaaggctcccttagtgataggccagtatagagaggcaggaaggctccccaaatccccaagctctggctctactgctccccaaactggaaagaccacttcctcacctcaacttgctttcagtagttcaggtgtgctcttcaaaggcacatgggaggggagggggcaggggcagggacagggaggctgggggtggatttgtaacctaggactttttttttctccaaccctcactgggagatggtggggcagtgctcccagctctgaaaagtaaagaaaccacacacttaatccctaagttttaaggggaacccacagcatactcctggaaagctagctttccttctaagaggcctggcagtggccccagggacccacagtgaggcaggccagaatgtgggtggctccccaccctcacctggggcaggctgcttctcctctcctcccccgcctggggaatcctagacaacacagacactgtatcaccccaaggctccaccagcctctccttttcaagggcagaagctgcaaaggaaagctcaggaggagaaaaagaaagttctgggagtgaaagaaaaagagccacagtggcaaaaccaagatgccttcataggctggcaagcaggggctgcttgtaccaggggcctccaaggttcccgttccagtggagaaacagacaccgggcaaagaccaaaagcaggtagtgacatctgtcaggttaatgctgtgaatgggccagagtcagggacaggacaggagtgggaacagaacaggagaaggctgcacactacatacagtggttaggggaggcctcattaaaacccatgcaaagttcccagggtagaaacacacagtgccagcctcagtcaggaggctggtcctacgggttcctcccctaccaggtgacccatgcctctcaaatgcacagcccctggtagactaggggtcctggtatacattctccaggcccagagctgcagccttcataaagaagcatacacttgtgccaagtcacaccaagagtgatgttaggggttaagccccatggcccagcttgcgcggccgcaggtgtgtcttccagtcagtcagtcagtgaaagtcctctccactgactgtagcctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacagg

SEQ ID NO: 32 - DNA, 5948 bp, Artificial Sequence
  Description of Artificial Sequence Synthetic polynucleotide
tttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctccttcctcttccagcccttcctctttccagtcagtcagtcagtggagactgtcgtgcggccgcgtggccttcatggctgtcctgcgctttccagggtcagagtgagttgactgcaccaatataggtaacttccagaatacacagagtaggtgctacatccagacacagagccaccacagccaacagtcacagaaggggtgggaggtccctgaacttacctgggtcacctgtcctagtgcagaaggatgctaagcttcaaacccaagcatcatcctcatctgcccttgtttgtgtgtttgattgagacacaacgtcctgctcagctaagctacagtcagtttgcattggcctctaagcctgggaagcatttccttggctgatgattggtgggggagggtacaggtcactgtgggcagttcagtgcctgggctgatggtcctgggtgctacaagagagcaatctgaacaaccaatggagagcaaaccactaagcagtttagttcttgactctgggttcctgccttgaggtcctgccctgactttactcagaggtggggcatgacttgagtattgtctgatcaagtatactgactgattcatccccaagctgttcttggtcatggagtttttaagacggcaatataaataccttaaaacaccttgtctctcactgggaccaggggctgggcaatttgactgcaggcagccagcaagcctcacagatccctgtgtctctaactccacggtcctgggattatagacatgtgacgccccgcacctaactttcttcatggatggaagtcaaatcctcctggttacgaggtaagtacatcaccaatggaggcatctccccagttcctgtatatttttaagacggtctttatagctcaggttggcctggaacttgtggcaacactcctgcctcagcctccagaatgctaggattgtaggtgtgaaccaccatccctggctaaacatgccatgttttacagttcaggatctggaagggaggtgattcccagcaagggctgcttcccaaggcacctcatcagcctgcctcaggtataacttcgtatagcatacattatacgaagttatactctacagaagaaatcctgttccccgcgcctccccataggagcaggagaaaaacagaggtttacccatgtgcaggatccagggcaatggcccttggctggtccaggtcctgccagaagagaaccttacgggacgtcccattgaggttggcaacctcaatgcggttggtctcggagtccgtccagtacagcttcttgccaacccagtcacaggccaggccatcaggtgacacgaggcccgagatgacaatgttctgtgcagcagctccagtctggttcaggtaggtctgtttgatggcctcctcgctcacatctgtccagtacacagcacccttggagaactggaagtctacagcagctgcatcctccaggccactggccacaatggtggactccagcttcactccgccggcatccactagccgcacatcccggcggttggcaaacaacaggagcggtgaggctgtgggcagagcacagggtcaggatggctaagcaggacaggaaccagctcccagggaaagagaatcatgcacggataacttcgtatagcatacattatacgaagttatctaccccgggcccctgctcccaagactccttccttccagttgcccaaaggctcccttagtgataggccagtatagagaggcaggaaggctccccaaatccccaagctctggctctactgctccccaaactggaaagaccacttcctcacctcaacttgctttcagtagttcaggtgtgctcttcaaaggcacatgggaggggagggggcaggggcagggacagggaggctgggggtggatttgtaacctaggactttttttttctccaaccctcactgggagatggtggggcagtgctcccagctctgaaaagtaaagaaaccacacacttaatccctaagttttaaggggaacccacagcatactcctggaaagctagctttccttctaagaggcctggcagtggccccagggacccacagtgaggcaggccagaatgtgggtggctccccaccctcacctggggcaggctgcttctcctctcctcccccgcctggggaatcctagacaacacagacactgtatcaccccaaggctccaccagcctctccttttcaagggcagaagctgcaaaggaaagctcaggaggagaaaaagaaagttctgggagtgaaagaaaaagagccacagtggcaaaaccaagatgccttcataggctggcaagcaggggctgcttgtaccaggggcctccaaggttcccgttccagtggagaaacagacaccgggcaaagaccaaaagcaggtagtgacatctgtcaggttaatgctgtgaatgggccagagtcagggacaggacaggagtgggaacagaacaggagaaggctgcacactacatacagtggttaggggaggcctcattaaaacccatgcaaagttcccagggtagaaacacacagtgccagcctcagtcaggaggctggtcctacgggttcctcccctaccaggtgacccatgcctctcaaatgcacagcccctggtagactaggggtcctggtatacattctccaggcccagagctgcagccttcataaagaagcatacacttgtgccaagtcacaccaagagtgatgttaggggttaagccccatggcccagcttgcgcggccgcaggtgtgtcttccagtcagtcagtcagtgaaagtcctctccactgactgtagcctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacagg

SEQ ID NO: 33 - DNA, 605 bp, Artificial Sequence
  Description of Artificial Sequence Synthetic polynucleotide
ataacttcgtataatgtatgctatacgaagttatccgtgcatgattctctttccctgggagctggttcctgtcctgcttagccatcctgaccctgtgctctgcccacagcctcaccgctcctcctctttgctaaccgccgagatgttcgactcgtagacgctggcggcgtgaagctggagagtacaatcgttgctagtggactggaggatgctgccgctgtcgacttccagttctcaaagggagccgtttattggaccgacgtgagcgaagaggcaataaaacagacataccttaaccagactggagctgccgcccagaatatcgtaatctctggcttggtgagccctgatggactcgcttgtgattgggtcggaaaaaagctttattggactgatagtgaaacgaatcggatagaagtagcgaaccttaacggtacatcaagaaaggtcctgttttggcaggatcttgaccagcctagagcaatcgccctcgaccctgcacatgggtaaacctctgtttttctcctgctcctatggggaggcgcggggaacaggatttcttctgtagagtataacttcgtataatgtatgctatacgaagttat

SEQ ID NO: 34 - DNA, 605 bp, Artificial Sequence
  Description of Artificial Sequence Synthetic polynucleotide
ataacttcgtataatgtatgctatacgaagttatccgtgcatgattctctttccctgggagctggttcctgtcctgcttagccatcctgaccctgtgctctgcccacagcctcaccgctcctgttgtttgccaaccgccgggatgtgcggctagtggatgccggcggagtgaagctggagtccaccattgtggccagtggcctggaggatgcagctgctgtagacttccagttctcaaagggagccgtttattggaccgacgtgagcgaggaggccatcaaacagacctacctgaaccagactggagctgctgcacagaacattgtcatctcgggcctcgtgtcacctgatggcctggcctgtgactgggttggcaagaagctgtactggacggactccgagaccaaccgcattgaggttgccaacctcaatgggacgtcccgtaaggttctcttctggcaggacctggaccagccaagggccattgccctggatcctgcacatgggtaaacctctgtttttctcctgctcctatggggaggcgcggggaacaggatttcttctgtagagtataacttcgtataatgtatgctatacgaagttat

SEQ ID NO: 35 - DNA, 605 bp, Artificial Sequence
  Description of Artificial Sequence Synthetic polynucleotide
ataacttcgtataatgtatgctatacgaagttatccgtgcatgattctctttccctgggagctggttcctgtcctgcttagccatcctgaccctgtgctctgcccacagcctcaccgctcctgttgtttgccaaccgccgggatgtgcggctagtggatgccggcggagtgaagctggagtccaccattgtggccagtggcctggaggatgcagctgctgtagacttccagttctccaagggtgctgtgtactggacagatgtgagcgaggaggccatcaaacagacctacctgaaccagactggagctgctgcacagaacattgtcatctcgggcctcgtgtcacctgatggcctggcctgtgactgggttggcaagaagctgtactggacggactccgagaccaaccgcattgaggttgccaacctcaatgggacgtcccgtaaggttctcttctggcaggacctggaccagccaagggccattgccctggatcctgcacatgggtaaacctctgtttttctcctgctcctatggggaggcgcggggaacaggatttcttctgtagagtataacttcgtataatgtatgctatacgaagttat

SEQ ID NO: 36 - DNA, 104 bp, Artificial Sequence
  Description of Artificial Sequence Synthetic polynucleotide
ggtgcggctagtggatgccgggttttagagctagaaatagcaagttaaaataaggctagtccgttatcaacttgaaaaagtggcaccgagtcggtgcttttttt

SEQ ID NO: 37 - DNA, 104 bp, Artificial Sequence
  Description of Artificial Sequence Synthetic polynucleotide
ggagtccaccattgtggccaggttttagagctagaaatagcaagttaaaataaggctagtccgttatcaacttgaaaaagtggcaccgagtcggtgcttttttt

SEQ ID NO: 38 - DNA, 104 bp, Artificial Sequence
  Description of Artificial Sequence Synthetic polynucleotide
ggtactggacagatgtgagcggttttagagctagaaatagcaagttaaaataaggctagtccgttatcaacttgaaaaagtggcaccgagtcggtgcttttttt

SEQ ID NO: 39 - DNA, 103 bp, Artificial Sequence
  Description of Artificial Sequence Synthetic polynucleotide
gctgaagatgaactgccagagttttagagctagaaatagcaagttaaaataaggctagtccgttatcaacttgaaaaagtggcaccgagtcggtgcttttttt

SEQ ID NO: 40 - DNA, 103 bp, Artificial Sequence
  Description of Artificial Sequence Synthetic polynucleotide
gatctccgtgaaggactcacgttttagagctagaaatagcaagttaaaataaggctagtccgttatcaacttgaaaaagtggcaccgagtcggtgcttttttt

SEQ ID NO: 41 - DNA, 103 bp, Artificial Sequence
  Description of Artificial Sequence Synthetic polynucleotide
gaccactggggaccagtcaagttttagagctagaaatagcaagttaaaataaggctagtccgttatcaacttgaaaaagtggcaccgagtcggtgcttttttt

SEQ ID NO: 42 - DNA, 104 bp, Artificial Sequence
  Description of Artificial Sequence Synthetic polynucleotide
gaggcgtttgccagagttcaggttttagagctagaaatagcaagttaaaataaggctagtccgttatcaacttgaaaaagtggcaccgagtcggtgcttttttt

SEQ ID NO: 43 - DNA, 4206 bp, Artificial Sequence
  Description of Artificial Sequence Synthetic polynucleotide
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggactga

SEQ ID NO: 44 - DNA, 2126 bp, Artificial Sequence
  Description of Artificial Sequence Synthetic polynucleotide
cgtctgtcccctagtaccatccacagagcagacattctctgtaagggtgtcttgcccttttgtgacctagagacgggatttggtgagcagccgtggtgatgttgctgtaccacactgcttctgtgtgctgtcagcgatggcatgtggaagagtttgcctgtgtcaaggtgctaagtcctaggctgccaggtcctgagtggttgggctcctttccagccatggttggttttttcttgctgtttgtcatcacctcctggggagagaacccagcactttatttttggggaaggaagtaaccttgcctcgttagaactgttgcattggctagccagagttcatctggagacacgagcctaggaggccacgggcacagcagacagtgattgccctgggcaccagctagagcctcctggactacttcacagatgctgcagcagggtggtgtttggggctcggttctgtcagctgactcacttctcagacacccccaaatcctaaataaataatagaattagcaggaagaagtatttgagaggttcataaaatttgccaggttttcacaaagtagccagacctatgttgttttcttcctttcttcatacaaggaaggtgacgttaaaccattaactcatctgcatggcgcactgtaaaatgacagttaagggcccagccctttcttggggactctggctcacttctgccagtggtgctgactcttcagttcccactgttaacagatggagtgttggggttgggatcatcctgcctgcctgacactcaggcagtatcccagtctccgttgcagtgtatttagcagtggtaaacgattgaaagcattcctacactgtttggttcagattggggcgaatctgtgtcttcctgggaacgtgatgtcatggccagttgcttctgcagagcttagaggaagggttgagctggtgcagtggcttctgtcataacttcgtataatgtatgctatacgaagttatgggctttgataatctgttaagatttgtttctgacttctctcgcagctccctccatacagcgggactctttgtagcatccaggctgaagacgaactgccagatggtaagccgggttgcatggactgggtgggcaggaaacctgcaaacatgtccataacttcgtataatgtatgctatacgaagttataacattgggtcatcttttctcaacacaaatttttatttattcatttattttggagtgtgcttttgtgtggaggtcagaggtcagctctctggaatatgtcctcttctgcttttgcctgggtttggtgattggacagaggtcatcgggcttatgcagcaagtctcttgctgagctgtctccctgatctgtccctgttgactcccatcctttgtaggttagcaggctcaggcttgtgcccagagctgcagtgtccgttcagccggaggcgcatttcattagagcttgaagcttctgctctgacctgcagctcacttaactcctgtcttacagtttccattgctgtggagagacaccatgaccagagcagccagtatgaaggcaaacatttatttggggctggtttacaggttcagaggttcagtccgttatcatggagggaaacatggcagcatccgggcatgcatggcactgggagctgagaattctctatgttgttctcaaggcaaacaggagaagtcttgcaagcagtgaggagggtctcatagcccacccgcacagtgccagttcctctaacaaggccacacctacccacagtgcctcggcccatgggccaagcatactcaaaccaccacaactgccaagtcactcagattggcaggtgtgctttcaaggttgtgccacgctcactctgggcatggtaagttgtggtcaggagagaggtgctctagtctcctcagcaggccctggtgttgtgggtgtgtcatcccacatggctgtgctcaactctgccgggtccagggagttgttatagcagttggtgttcatgacccatgcctgggcatttaggcctccagcatgagccctggccgatttatgcataagtgtcatgtttatcaaggtgcagcagtgtttgttactgaggtgtgtttaagatatcctggctatgcagt

SEQ ID NO: 45 - DNA, 1832 bp, Artificial Sequence
  Description of Artificial Sequence Synthetic polynucleotide
gaaagtttaattctactcttatttgaaaggaatgtttgtttactcatgaagtaaaggaccccccaatattaagaactgggtgatttgattattaattaatctcagtgttttaaattttaatatatatacatctaagcatatgtgtacatggtctatgcaggtacatgcatggggccagaagagggcaccagatgtatgcttccatcactctctccacctattcttgtggggcagcgtcccctcctaaacctggggcttgtgtgttgactagactggaagccagaaagcctcagagagcctctcctctctgctcctctcagagatgggagcataggtatttgtgagatgcctgacttgttacacaggttctgggacctgaatgctggccctcatctctggaccatcactccaggccttaaaatcagttgttacacaacaacaacgacaacaccaccaccaccacaacaaaaatggcacaatcccagtaaagacaataacttcgtataatgtatgctatacgaagttatctataacttcagctctgcctgttcccttttgatgcctgattccctgcccctcttctgtcctaggtgctgtaaaaggagaactcctcattgacatcggctctggcccaaccatctaccagcttctgtctgcctgtgagtctttcacggagatcatcgtctctgactatacagaccaaaacctctgggaactgcagaaatggctcaagaaggagccaggagcctttgattggtccccagtcgtcacctatgtgtgcgatcttgaaggcaacaggtagaggaatgagtatctgctcttcaactttctgaagggacctgcataacttcgtataatgtatgctatacgaagttatatgtttctatcagcccaaactaccactaagatccagatgaaaaatgcaaaagaaactcaaagaccaaagggaactcgggatagaagatgagggctcaggagaatagctgggttgtcagatgtaccaccagagtactatttggttaccagtcatccaacacaaaggtggtggctaatgcctaatgcccacaccaccggaaacagcaaaacactggcagatgtatttttttagccaacagaaataacctgaagcaattatggggaaagaattcattacttaccatctataaagttagaatctttaggatctgggagacggtcagtgggtaaagtgcttgtcacagaggttgtcctttctccagtgtgtcctcttagcatctttattgaaaaccagctggctgcagattcatggcttagtcctatactgtctattctgttccattattctgtgttctctggctggtgatgttttgtggaaagactttgctggtttgatgatatagtttttttgttgttgttgttgctgctgctgctgctgttgttgaagctaagtagtgtgtgatttctgatattttcttggtgctatttgggctttgtgtgtacggttgcctaaaaattctagaattgttttctagtcctgtcattagtattttaagaggaatggcattggctacactaattgctttgggtagtatggacatattcatgatcttgattcttcagatcagcgaacagatctttccattttgatggtgtctgcactattcttctccattgatgttttcagtgtaaagatcattggcttgttttagcaaaattcattttgaatttatatattatatttattaataaatacattaataaaataatattaaatattacattttatttatttttaatagctattgcaataggattgacttcttgatttctgaatcaaataattctctattggg

SEQ ID NO: 46 - DNA, 1431 bp, Artificial Sequence
  Description of Artificial Sequence Synthetic polynucleotide
gacagaggcaggtaaattactgtgaattccagtctagccagcgatatgtagtgagaccctgcctaaaatatattaaaaaaaaaaaaaaaaagggaaggaagagtaaatggatttgtctgatagtctgtctggcacgagtgttgtttgataaacgcatcttgtgataacttcgtataatgtatgctatacgaagttatttatctgtctggcattgccatgcttttataccgtcccgaccacacatcttcccacaggtgcctgccaggctgggttggtgagcgctgccagctcgaagacccctgccactccggcccttgtgccggccgaggcgtctgccagagctcagtggtggctggcaccgcacgattctcctgccggtgccttcgaggcttccaaggtgaaggggtgtgtctggacgggaaccctataacttcgtataatgtatgctatacgaagttattggtaggcgagaatgtagtcagacccaagctcaccctctcctggttcttccaggcccagactgctcccagccagacccctgcgtcagcaggccctgtgttcatggtgccccctgctcagtggggccggatggccgatttgcctgtgcctgcccacctggctaccagggtcaaagctgccaaagtgacatagatgagtgccgatctggtacaacttgccgtcatggtggtacctgtctcaatacacctggatccttccgctgccagtgtcctcttggttatacagggctgctgtgtgagaaccccgtagtgccctgtgccccttccccgtgtcgtaatggtggcacctgtaggcagagcagtgatgtcacatatgactgtgcttgccttcctggtaagtaagttgtgcccagggaaggcagctggggacaataggctagcctcttagtgaccattgtcaccttgtcctcccctacgaggcttcgagggccagaactgtgaagtcaacgtggatgactgtcctggacatcggtgtctcaatgggggaacgtgtgtagacggtgtcaatacttacaactgccagtgccctccggagtggacaggtgggcatcagggctgcagagaaccagggtggctgacctcaggtgggcacacgggcaacttagactagcacatctttgtgccctaggccagttctgtacagaagatgtggatgagtgtcagctgcagcccaatgcctgccacaatgggggtacctgcttcaacctactgggtggccacagctgtgtatgtgtcaatggctggacgggtgagagctgcagtcagaatatcgatgactgtgctacagccgtgtgtttccatggggccacctgccatgaccgtgtggcctctttctactgtgcctgccctatggggaagacaggtgagtggcccttttctttgtaggcaacagaatggtttcagcatgaaaggt